(19) United States
(12) Patent Application Publication
(10) Pub. No.: US 2008/0070836 A1
(43) Pub. Date: Mar. 20, 2008

(54) Emt-Inducing Agents

(75) Inventors: Toshio Hirano, Osaka (JP); Susumu Yamashita, Osaka (JP)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Toshio Hirano, Osaka-shi, Osaka (JP)

(21) Appl. No.: 10/584,742

(22) PCT Filed: Dec. 22, 2004
(86) PCT No.: PCT/JP04/19246

§ 371 Date: Apr. 23, 2007

(30) Foreign Application Priority Data
Dec. 26, 2003 (JP) 2003-435122

(51) Int. Cl. A61K 38/16; A61K 31/7052; A61P 17/02; A61P 29/00; A61P 35/00; C07H 21/00; C07K 14/00; C12N 15/63; C12Q 1/68

(52) U.S. Cl. 514/12; 435/320.1; 435/6; 514/44; 530/350; 536/23.1; 536/23.5

(57) ABSTRACT

The mechanism of STAT3, which is considered to play a crucial role in EMT, was elucidated using zebrafish embryos. Unexpectedly, a STAT3 target gene turned out to be zinc transporter LIV1. The present inventors studied the relationship between STAT3 and LIV1 in EMT, and further studied their relationship with zinc finger protein Snail, known for its association with EMT. The results showed that LIV1, whose expression is regulated by STAT3, activated Snail, thereby ultimately inducing EMT. LIV1 can be used as an EMT regulatory agent. Further, because EMT is involved in cancer progression, LIV1 antisense nucleotides and the like may be used as pharmaceuticals for treating cancer.

### TECHNICAL FIELD

This invention relates to the uses of LIV1 for regulating epithelial-mesenchymal transition (EMT).

### BACKGROUND ART

Epithelial-mesenchymal transition (EMT) is a central event in embryonic development, organ and tissue regeneration, and tumor metastasis and progression. Epithelial-mesenchymal transition (EMT) is a phenotypic cell transition where, in the processes of gastrulation, wound healing, tumor progression, and the like, epithelial cells disperse by weakening the cell-cell adhesion system, and show invasive cell behavior as mesenchymal cells (Non-Patent Document 1). To understand development and tumor metastasis and progression, the mechanism of EMT must be elucidated; however, much regarding EMT remains unknown.

There are a few reports of evidence that indicates a relationship between EMT and the changes in the cell-cell adhesion system caused by Snail and Slug. Snail and Slug are zinc-finger proteins that change the cell-cell adhesion system. Snail family proteins were first reported as substances playing an important role in Drosophila gastrulation. Snail has recently come to be considered important as a direct repressor of the transcription of cell-cell adhesion molecules such as E-cadherin, which is involved in adhesive binding, and claudins and occluding, which are involved in tight junctions (Non-Patent Documents 1-4). Snail and Slug show similar localization patterns in the embryos of mice, chickens, Xenopus, zebrafish, and Drosophila. Loss of Snail or Slug function in these embryos results in defective gastrulation and/or neural crest migration (Non-Patent Documents 1, 5-7). Further, enhanced Snail expression is thought to correlate with dedifferentiation and acquirement of metastatic potential in many human cancers. Thus, the potential role of Snail and Slug in the physiological or pathological in vivo processes of EMT can be considered to be evolutionarily conserved (Non-Patent Document 1). However, mechanisms for regulating the activity of expressed Snail remain unknown.

On the other hand, signal transducers and activators of transcription (STAT) are transcription factors that respond to a variety of cytokines and growth factors, and mediate their biological functions (Non-Patent Documents 8-10). For example, they mediate biological functions such as cell proliferation, differentiation, and survival. Furthermore, STATs are involved in vertebrate gastrulation and wound healing, as well as in cancer metastasis in vertebrate animals and cell movement in similar processes in Drosophila and Dictyostelium discoideum (Non-Patent Document 11). Previously, the present inventors revealed that STAT3 is activated in the organizer during the gastrulation process in zebrafish, and that STAT3 activity is essential for gastrulation movements, but is not required for initial cell developmental fate specificity. This requirement for STAT3 is cell autonomous for the anterior migration of gastrula organizer cells, and non-cell-autonomous for convergence of neighboring cells (Non-Patent Document 12). In addition, STAT is required for cell migration but is not required for cell proliferation, with respect to the border cell migration that occurs during oogenesis in Drosophila (Non-Patent Document 13), chemotaxis of Dictyostelium discoideum (Non-Patent Document 14), and dermal wound healing processes in mice (Non-Patent Document 15). Furthermore, constitutive activation of STAT family members, and of STAT3 in particular, is observed in many human cancers. This fact means that the above STAT family members are involved in cell growth and survival (Non-Patent Document 16). However, it is thought that STAT3 may also influence cell-cell adhesion and cancer cell movement. Based on the above observations, it is highly likely that the role of STAT signal transduction in EMT is evolutionarily conserved throughout those processes. However, the entire molecular mechanism of STAT function in EMT is unknown.
[Non-Patent Document 1] Thiery, J. P. Epithelial-mesenchymal transitions in tumour progression. Nat Rev Cancer 2, 442-54 (2002).
[Non-Patent Document 2] Batlle, E. et al. The transcription factor snail is a repressor of E-cadherin gene expression in epithelial tumor cells. Nat Cell Biol 2, 84-9 (2000).
[Non-Patent Document 3] Cano, A. et al. The transcription factor snail controls epithelial-mesenchymal transitions by repressing E-cadherin expression. Nat Cell Biol 2, 76-83 (2000).
[Non-Patent Document 4] Ikenouchi, J., Matsuda, M., Furuse, M. & Tsukita, S. Regulation of tight junctions during the epithelium-mesenchyme transition: direct repression of the gene expression of claudins/occludin by Snail. J Cell Sci 116, 1959-67 (2003).
[Non-Patent Document 5] Leptin, M. twist and snail as positive and negative regulators during Drosophila mesoderm development. Genes Dev 5, 1568-76 (1991).
[Non-Patent Document 6] Nieto, M. A., Sargent, M. G., Wilkinson, D. G & Cooke, J. Control of cell behavior during vertebrate development by Slug, a zinc finger gene. Science 264, 835-9 (1994).
[Non-Patent Document 7] Carver, E. A., Jiang, R., Lan, Y, Oram, K. F. & Gridley, T. The mouse snail gene encodes a key regulator of the epithelial-mesenchymal transition. Mol Cell Biol 21, 8184-8 (2001).
[Non-Patent Document 8] Bromberg, J. & Darnell, J. E., Jr. The role of STATs in transcriptional control and their impact on cellular function. Oncogene 19, 2468-73 (2000).
[Non-Patent Document 9] Darnell, J. E., Jr. STATs and gene regulation. Science 277, 1630-5 (1997).
[Non-Patent Document 10] Hirano, T., Ishihara, K. & Hibi, M. Roles of STAT3 in mediating the cell growth, differentiation and survival signals relayed through the IL-6 family of cytokine receptors. Oncogene 19, 2548-56 (2000).
[Non-Patent Document 11] Yamashita, S. & Hirano, T. in Signal Transducers and Activators of Transcription (STATs): Activation and Biology (eds. Sehgal, P. B., Hirano, T. & Levy, D. E.) (Kluwer Academic Publishers, Dordrecht, The Netherlands, in press).
[Non-Patent Document 12] Yamashita, S. et al. Stat3 Controls Cell Movements during Zebrafish Gastrulation. Dev Cell 2, 363-75 (2002).
[Non-Patent Document 13] Silver, D. L. & Montell, D. J. Paracrine signaling through the JAK/STAT pathway activates invasive behavior of ovarian epithelial cells in Drosophila. Cell 107, 831-41 (2001).
[Non-Patent Document 14] Mohanty, S. et al. Evidence that the Dictyostelium Dd-STATa protein is a repressor that regulates commitment to stalk cell differentiation and is also required for efficient chemotaxis. Development 126, 3391-405 (1999).
[Non-Patent Document 15] Sano, S. et al. Keratinocyte-specific ablation of Stat3 exhibits impaired skin remodeling, but does not affect skin morphogenesis. Embo J 18, 4657-68 (1999).
[Non-Patent Document 16] Bowman, T., Garcia, R., Turkson, J. & Jove, R. STATs in oncogenesis. Oncogene 19, 2474-88 (2000).
[Non-Patent Document 17] Manning, D. L., Daly, R. J., Lord, P. G., Kelly, K. F. & Green, C. D. Effects of oestrogen on the expression of a 4.4 kb mRNA in the ZR-75-1 human breast cancer cell line. Mol Cell Endocrinol 59, 205-12 (1988).
[Non-Patent Document 18] Manning, D. L. et al. Oestrogen-regulated genes in breast cancer: association of pLIV1 with lymph node involvement. Eur J Cancer 30A, 675-8 (1994).
[Non-Patent Document 19] Taylor, K. M. & Nicholson, R. I. The LZT proteins; the LIV-1 subfamily of zinc transporters. Biochim Biophys Acta 1611, 16-30 (2003).
[Non-Patent Document 20] Taylor, K. M., Morgan, H. E., Johnson, A., Hadley, L. J. & Nicholson, R. I. Structure-function analysis of LIV-1,the breast cancer-associated protein that belongs to a new subfamily of zinc transporters. Biochem J 375, 51-9 (2003).
[Non-Patent Document 21] Nasevicius, A. & Ekker, S. C. Effective targeted gene ‘knockdown’ in zebrafish. Nat enet 26, 216-20 (2000).
[Non-Patent Document 22] Kozlowski, D. J. & Weinberg, E. S. Photoactivatable (caged) fluorescein as a cell tracer for fate mapping in the zebrafish embryo. Methods Mol Biol 135, 349-55 (2000).
[Non-Patent Document 23] Thisse, C., Thisse, B., Schilling, T. F. & Postlethwait, J. H. Structure of the zebrafish snaill gene and its expression in wild-type, spadetail and no tail mutant embryos. Development 119, 1203-15 (1993).
[Non-Patent Document 24] Thisse, C., Thisse, B. & Postlethwait, J. H. Expression of snail2, a second member of the zebrafish snail family, in cephalic mesendoderm and presumptive neural crest of wild-type and spadetail mutant embryos. Dev Biol 172, 86-99 (1995).
[Non-Patent Document 25] Solnica-Krezel, L., Stemple, D. L. & Driever, W. Transparent things: cell fates and cell movements during early embryogenesis of zebrafish. Bioessays 17, 931-9 (1995).
[Non-Patent Document 26] Blanco, M. J. et al. Correlation of Snail expression with histological grade and lymph node status in breast carcinomas. Oncogene 21, 3241-6 (2002).
[Non-Patent Document 27] Fujita, N. et al. MTA3, a Mi-2/NuRD complex subunit, regulates an invasive growth pathway in breast cancer. Cell 113, 207-19 (2003).
[Non-Patent Document 28] Peinado, H., Quintanilla, M. & Cano, A. Transforming growth factor beta-1 induces snail transcription factor in epithelial cell lines: mechanisms for epithelial mesenchymal transitions. J Biol Chem 278, 21113-23 (2003).

### DISCLOSURE OF THE INVENTION

### Problems to be Solved by the Invention

An objective of the present invention is to provide EMT regulatory agents and novel pharmaceuticals for treating cancer.

### Means to Solve the Problems

To solve the above problems, the present inventors used zebrafish embryos to elucidate the molecular mechanism of STAT3 function, considered to be critical to EMT. The present inventors isolated a STAT3 target gene, which was unexpectedly found to be LIV1.

Thus, the present inventors continued to study LIV1. First, they revealed that the LIV1 gene was expressed in gastrula organizer cells in zebrafish. Next, they examined the effect of suppressing LIV1 expression on embryos, by specifically suppressing LIV1 gene expression in several kinds of zebrafish embryos. The results indicated that defective LIV1 activity in zebrafish embryos interferes with the extent of organizer cell migration. By expressing the LIV1 gene in the organizer cells of zebrafish embryos with inhibited STAT3 expression, the present inventors also revealed that, of the defective STAT3 functions, cell autonomous functions could be rescued by LIV1 gene expression, but non-cell-autonomous functions could not. Further, they showed that LIV1 enhanced the repressor activity of the zinc finger protein Snail, which is expressed independently of STAT3 and LIV1 activities, and also showed that Snail was essential for LIV1 function in EMT. These results indicated that LIV1 is an essential and sufficient STAT3 target gene for STAT3's cell autonomous role in anterior migration of organizer cells. The three molecules, STAT3, LIV1, and Snail, were also confirmed to have some relationship regarding the EMT process of gastrula organizer cells in zebrafish embryos.

LIV1 was initially identified as a breast cancer protein whose expression was regulated by estrogen. Although LIV1 was known to be involved in the expansion of cancer metastasis (Non-Patent Documents 17 and 18), it was recently revealed that LIV1 belongs to the ZIP zinc transporter subfamily (Zrt-, Irt-like proteins), termed LZT (an LIV-1 subfamily of ZIP zinc transporters) (Non-Patent Document 19), and that LIV1 functions as a zinc transporter protein (Non-Patent Document 20).

Based on the above findings, the present inventors established the following model EMT mechanism. The expression of zinc finger protein Snail is regulated by MAPK through TGF-β or FGF (Non-Patent Document 28). The zinc transporter LIV1, whose expression is regulated by STAT3, activates the zinc finger protein Snail, which leads to down-regulation of the cell-cell adhesion system, and ultimately induces EMT. The above model EMT mechanism is shown in FIG. 5r.

LIV1 and Snail are reported to be involved in the expansion of breast cancer metastasis (Non-Patent Documents 1, 18, 26, and 27). In addition, STAT3 is constitutively activated in many tumors including breast cancer. Thus, similar mechanisms may contribute to cancer progression (Non-Patent Document 16). However, nothing was known regarding the role of LIV1 in vivo and whether or not it was critical for cancer cell metastasis. Herein, the present inventors demonstrated for the first time that LIV1 contributes to EMT, and is further involved in cancer metastasis. Specifically, the present inventors elucidated the mechanism of Epithelial-mesenchymal transition (EMT) related to cancer metastasis and progression, and provided the EMT regulatory agents, pharmaceuticals for treating cancer, and the like, of this invention. More specifically, the present invention provides the following:

[1] A regulatory agent of Snail activity, which is an isolated DNA of any one of the following (a) to (d):

(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

[2] A regulatory agent of Snail activity, which is a vector into which a DNA of any one of the following (a) to (d) is inserted:

(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

[3] A regulatory agent of Snail activity, which is an isolated protein encoded by a DNA of any one of the following (a) to (d):

(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

[4] An agent for suppressing Snail activity, which is an antisense oligonucleotide targeting a DNA sequence comprising the sequence of SEQ ID NO: , 4, 25, 27, 29, 31, 33, 35, 37, 39, or 41.

[5] An agent for suppressing Snail activity, which is a double-stranded RNA comprising a sequence identical or similar to a portion of a DNA comprising the sequence of SEQ ID NO: 3, 4, 25, 27, 29, 31, 33, 35, 37, 39, or 41.

[6] A pharmaceutical for treating cancer, which comprises a nucleotide or vector of any one of the following (a) to (c), as an active ingredient:

(a) an antisense oligonucleotide of a DNA comprising the sequence of SEQ ID NO: 3;
(b) a vector into which an antisense oligonucleotide of a DNA comprising the sequence of SEQ ID NO: 3 is inserted; and
(c) an oligonucleotide that is a double-stranded RNA comprising a sequence identical or similar to a portion of a DNA comprising the sequence of SEQ ID NO: 3.

[7] A pharmaceutical for treating cancer, which comprises the agent for suppressing Snail activity of [4 or 5 as an active ingredient.

[8] An EMT-inducing agent, which is an isolated DNA of any one of the following (a) to

(d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

[9] An EMT-inducing agent, which is a vector into which a DNA of any one of the following (a) to (d) is inserted:

(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39,or 41.

[10] An EMT-inducing agent, which is an isolated protein encoded by a DNA of any one of the following (a) to (d):

(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

[11] An EMT-suppressing agent, which is an antisense oligonucleotide targeting a DNA sequence comprising the sequence of SEQ ID NO: 3, 4, 25, 27, 29, 31, 33, 35, 37, 39, or 41.

[12] An EMT-suppressing agent, which is a double-stranded RNA comprising a sequence identical or similar to a portion of a DNA comprising the sequence of SEQ ID NO: 3, 4, 25, 27, 29, 31, 33, 35, 37, 39, or 41.

[13] A pharmaceutical for treating cancer, which comprises the EMT-suppressing agent of [11] or [12] as an active ingredient.

[14] A method of screening for a candidate substance for an agent for suppressing Snail activity, wherein the method comprises the following steps of (a) to (c):

(a) injecting a vector which comprises a reporter gene operably linked under the control of the E-cadherin promoter, and a test substance into a one-cell-stage embryo;
(b) measuring the expression level of the reporter gene; and
(c) selecting a compound that reduces or increases the measured expression level of the reporter gene, compared with the measured expression level in the absence of the test substance.

[15] A wound healing agent, which comprises the regulatory agent of Snail activity of any one of [1] to [3], or the EMT-inducing agent of any one of [8] to [10], as an active ingredient.

[16] An anti-inflammatory agent, which comprises the agent for suppressing Snail activity of [4] or [5], or the EMT-suppressing agent of [11] or [12], as an active ingredient.

[17] A regulatory agent of a protein activity that requires zinc, which comprises a protein comprising the amino acid sequence of SEQ ID NO: 1, 2, 26, 28, 30, 32, 34, 36, 38, 40, or 42 as an active ingredient.

### BRIEF DESCRIPTION OF THE DRAWINGS

FIG. 1a shows a diagram of a zebrafish LIV1 protein and its putative amino acid sequence. The locations of eight predicted transmembrane domains (marked (3) in the figure), the CPALLY motif (marked (2) in the figure), and the histidine rich repeats (marked (1) in the figure) are shown. Roman numerals indicate the transmembrane domains. The underlined regions in the amino acid sequence shown by a number in parentheses correspond to the regions in the protein diagram indicated by the same number in parentheses.

FIG. 1b shows a comparison between zebrafish LIV1 (LZT-Zf3) and the human LZT subfamily of ZIP transporter proteins (LZT-H). The sequences are across the transmembrane domains IV and V, containing the conserved HNF motif and HEXPHE motif. Amino acids that correspond to those in the zebrafish LIV1 protein are emphasized (enclosed within a border).

FIGS. 1c-j show the expression of zebrafish LIV1 MRNA in normal embryos (FIGS. 1c-h), a STAT3D4 control morpholino-injected embryo (FIG. 1i), and a STAT3-morpholino-injected embryo (FIG. 1j) at the gastrula stage. FIGS. 1c-e, i and j show lateral views; FIGS. 1f and h show animal-pole views; and FIG. 1g shows a dorsal view. Dorsal is to the right in FIGS. 1c-f, i and j.

FIGS. 2a-d show the phenotypes one day after fertilization (FIGS. 2a and b; side views, with anterior to the left) and at the end of gastrulation (FIGS. 2c and d; lateral views, with dorsal to the right). The arrows in FIG. 2c and FIG. 2d indicate the anterior limit of the hypoblast (Polster). The black triangles indicate the posterior limit of the hypoblast (tail bud). FIGS. 2e-v show the expression of the various markers. FIGS. 2e-h and k-p show lateral views, with dorsal to the right, at the one-somite stage. FIGS. 2i-j and s-v show dorsal views at the one-somite stage. FIGS. 2q and r show animal-pole views at the one-somite stage.

FIG. 3 shows the results of cell-tracing experiments. FIG. 3a: axial mesendodermal cells in a normal control embryo; FIG. 3b: axial mesendodermal cells in a LIV1-depleted embryo; FIG. 3d: lateral mesendodermal cells in a normal control embryo; and FIG. 3e: lateral mesendodermal cells in a LIV1-depleted embryo. The animal pole is facing up, and dorsal is to the right in FIGS. 3a, b, d, and e. FIGS. 3c and f show graphs comparing anterior migration (FIG. 3c), and dorsal convergence (FIG. 3f) of labeled cell groups in the control (LIV1D4-MO), LIV1-depleted (LIV1-MO), and Stat3-depleted (Stat3-MO) embryos. The arrows indicate the leading edge of anterior migrating axial mesendodermal cells (FIG. 3c) and dorsal converging lateral mesendodermal cells (FIG. 3f); and the black triangles indicate the initial position at the shield stage.

FIG. 4a shows a scheme illustrating cell transplantation experiments. FIGS. 4b-m show the migration of prechordal mesendodermal cells obtained from predetermined morpholino- and mRNA-injected donors after being transplanted into the shield region of a predetermined morpholino-injected host at the early gastrula stage. All panels show the transplanted host embryos at the end of gastrulation. The donors (donor 1 and donor 2) and hosts in each panel were as follows:
FIG. 4b: donor 1: LIV1D4-MO, donor 2: LIV1-MO, host: LIV1D4-MO Host;
FIG. 4c: donor 1: LIV1D4-MO, donor 2: LIV1-MO+Liv1, host: LIV1D4-MO Host;
FIG. 4d: donor 1: LIV1D4-MO, donor 2: LIV1-MO, host: LIV1-MO Host;
FIG. 4e: donor 1: LIV1D4-MO, donor 2: LIV1-MO+LIV1, host: LIV1-MO Host;
FIG. 4f: donor 1: STAT3D4-MO, donor 2: STAT3-MO, host: STAT3D4-MO Host;
FIG. 4g: donor 1: STAT3D4-MO, donor 2: STAT3-MO+STAT3, host: STAT3D4-MO Host;
FIG. 4h: donor 1: STAT3D4-MO, donor 2: STAT3-MO+STAT5, host: STAT3D4-MO Host;
FIG. 4i: donor 1: STAT3D4-MO, donor 2: STAT3-MO+Liv1, host: STAT3D4-MO Host;
FIG. 4j: donor 1: STAT3D4-MO, donor 2: Snail1-MO, host: STAT3D4-MO Host;
FIG. 4k: donor 1: STAT3D4-MO, donor 2: Snail1-MO+Snail1, host: STAT3D4-MO Host;
FIG. 41: donor 1: STAT3D4-MO, donor 2: Snail1-MO+Liv1, host: STAT3D4-MO Host; and
FIG. 4m: donor 1: STAT3D4-MO, donor 2: STAT3 -MO+LiV1+Snail1-MO, host:
STAT3D4-MO Host.

Black triangles indicate the initial transplant position, and arrows indicate the leading edge of the anterior migrating transplanted cells. Dotted arrows indicate the migration of donor 1 cells, and solid arrows indicate the migration of donor 2 cells . FIG. 4n shows a scheme illustrating cell tracing experiments. FIGS. 4o-r show lateral mesendodermal cells in hosts (Host; circled area indicated by the dotted arrow) transplanted with prechordal mesendodermal cells (Donor; circled area indicated by the solid arrow) at the end of gastrulation. The donor and host in the each panel are as follows:
FIG. 4o: donor: STAT3-MO, host: STAT3-MO Host;
FIG. 4p: donor: STAT3-MO+STAT3, host: STAT3-MO Host;
FIG. 4q: donor: STAT3-MO+STAT5, host: STAT3-MO Host; and
FIG. 4r: donor: STAT3-MO+LiV1, host: STAT3-MO Host.

The arrows with black triangles indicate the leading edge of labeled cells dorsally converging from the initial position.

FIGS. 5a-d show the phenotypes of various morpholino-oligonucleotide-injected embryos at the end of the gastrulation stage (lateral views, with dorsal to the right). FIG. 5a: LIV1D4 control-morpholino-injected embryo; FIG. 5b: LIV1-morpholino-injected embryo; FIG. 5c: STAT3-morpholino-injected embryo; and FIG. 5d: Snaill-morpholino-injected embryo. The arrows indicate the anterior limit of the hypoblast. FIGS. 5e-h show the morphology of organizer cells in the anterior-most limit of the hypoblast in each embryo at the mid-gastrula stage. FIG. 5e: Control; FIG. 5f: LIV1-morphant; FIG. 5g: STAT3-morphant; and FIG. 5h: Snaill-morphant. FIGS. 5i-p show the expression of LIV1 (FIGS. 5i-l) and Snail1 (FIGS. 5m-p) in each embryo at the early gastrula stage (6 hpf.). FIGS. 5i and m: Control; FIGS. 5j and n: LIV1-morphant; FIGS. 5k and o: STAT3-morphant; and FIGS. 51 and p: Snail1-morphant. All panels in FIGS. 5i-p show animal-pole views.

FIG. 5q shows Snail-dependent LIV1 activity using the E-cadherin promoter activity as an indicator. Zebrafish LIV1 RNA (1, 10, or 100 pg/embryo; lanes 6-14); mouse Snail RNA (1, 10, or 100 pg/embryo; lanes 2-4 and 9-11); and/or zebrafish Snail1 morpholino (10 ng/embryo; lanes 5 and 12-14). FIG. 5r shows a model for the interaction between LIV1 and Snail in EMT.

The upper half of FIG. 6 shows photographs of the morphological changes in DU145 cells after using RNAi to silence the endogenous human LIV1 in DU145 cells. The lower half of FIG. 6 is the results of using RT-PCR to show the expression levels of LIV1 and E-cadherin in the cultured cells. shRNA-NC indicates cells transfected with control shRNA, and shRNA-Liv1#2 indicates cells where LIV1 is silenced by shRNA.

### BEST MODE FOR CARRYING OUT THE INVENTION

The present invention relates to LIV1, a downstream target of STAT3. As described above, the present inventors revealed that LIVl gene expression regulates EMT by influencing the activity of Snail. Therefore, the LIV1 protein can be used to regulate Snail activity and EMT.

Herein, regulatory agents of Snail activity are defined as substances capable of positively regulating the activity of the zinc finger protein Snail, directly or indirectly. By regulating Snail activity, the regulatory agents of Snail activity may be capable of changing cell adhesion and inducing epithelial-mesenchymal transition (EMT), or of affecting cancer metastasis and progression.

Preferable examples of substances that can be used as such regulatory agents of Snail activity may be isolated LIV1 proteins comprising the amino acid sequence of SEQ ID NO: 1 or 2. SEQ ID NOs: 3 and 4 show examples of DNA sequences encoding the LIV1 protein. In addition, proteins similar to LIV1 may be used as the regulatory agents of Snail activity of this invention. The proteins similar to LIV1 may preferably be isolated proteins of the human LZT subfamily, comprising the amino acid sequence of SEQ ID NO: 26,28, 30,32,34,36,38,40, or 42. DNA sequences encoding the human LZT subfamily are shown in SEQ ID NO: 25, 27, 29, 31,33,35,37,39, and 41. Herein, the term “LIV1 protein” includes proteins similar to LIV1 unless otherwise specifically stated.

Herein, the term “isolated” means that a substance is separated from its original environment (for example, from the natural environment, for a naturally occurring substance), and that the environment of the substance has been artificially changed. In addition, the term “isolation” means comprising a compound present in a sample where the compound of interest is substantially rich, and/or a compound present in a sample where the compound of interest is partially or substantially purified. Herein, the term “substantially purified” means that a compound is separated from its natural environment, and comprises less than 60% of other naturally associated components, preferably less than 75%, and most preferably less than 90%.

The above LIV1 proteins may be prepared by a variety of methods well known to those skilled in the art. For example, such a protein may be prepared by producing it in a transformant carrying a vector to which a DNA comprising the nucleotide sequence of SEQ ID NO: 3, 4, 25, 27, 29, 31, 33, 35, 37, 39, or41 has been inserted, and then purifying it. Vectors for such use may be appropriately selected depending on the translation system used for protein production. In addition, LIV1 is known to be expressed in hormonal tissues such as breast, prostate, hypophysis, and brain (Non-Patent Document19). Thus, LIV1 protein may be purified from cell extracts expressing LIV1, for example, from human and zebrafish cell extracts of the above hormonal tissues, by preparing an anti-LIV1 protein antibody according to known methods and creating an affinity column using the antibody.

The above-described proteins that are similar to LIV1 are capable of regulating the activity of Snail and may include, for example, proteins comprising an amino acid sequence with one or more amino acid substitutions, deletions, insertions, and/or additions in the amino acid sequence of SEQ ID NO: 1, 2, 26, 28, 30, 32, 34, 36, 38, 40, or 42, and proteins encoded by DNAs that hybridize under stringent conditions with a DNA comprising the nucleotide sequence of SEQ ID NO: 1.

Examples of methods that can be used to prepare the above proteins, which are similar to LIV1, include methods that carry out hybridization with a nucleotide sequence encoding LIV1 to obtain highly homologous DNAs, prepare transformants using these DNAs, and produce the desired proteins in those transformants. For example, a highly homologous DNA may be obtained from human or non-human vertebrate cells or such by performing hybridization under stringent conditions, using part of the nucleotide sequence of SEQ ID NO: 3, 4, 25, 27, 29, 31, 33, 35, 37, 39, or41 as a probe.

Those skilled in the art can appropriately select the above stringent hybridization conditions. For example, pre-hybridization is carried out in a hybridization solution containing 25% formamide, or 50% formamide under more stringent conditions, and 4x SSC, 50 mM Hepes (pH 7.0), 10x Denhardt's solution, and 20 μg/ml denatured salmon sperm DNA at 42° C. overnight. A labeled probe is then added to the solution and hybridization is carried out by incubation at 42° C. overnight. Post-hybridization washes are carried out at different levels of stringency, including the moderately stringent “1x SSC, 0.1% SDS, 37° C.”, highly stringent “0.5x SSC, 0.1% SDS, 42° C.”, and more highly stringent “0.2x SSC, 0.1% SDS, 65° C.” conditions. As the stringency of the post-hybridization washes increases, DNAs with greater homology to the probe sequence are expected to be isolated. The above-described combinations of SSC, SDS, and temperature are merely examples of washing conditions. Those skilled in the art can achieve the same stringencies as those described above by appropriately combining the above factors or others (such as probe concentration, probe length, or hybridization period) that affect hybridization stringency.

Polypeptides encoded by DNAs isolated using such hybridization techniques will usually comprise amino acid sequences highly homologous to the polypeptides identified by the present inventors. “High homology” refers to sequence homology of at least 40% or more, preferably 60% or more, further preferably 80% or more, further preferably 90% or more, further preferably at least 95% or more, and further preferably at least 97% or more (for example, 98% to). Amino acid sequence identity can be determined, for example, using the BLAST algorithm of Karlin and Altschul (Proc. Natl. Acad. Sci. USA. 87:2264-2268, 1990; Proc. Natl. Acad. Sci. USA 90: 5873-5877, 1993). A program called BLASTX has been developed based on this algorithm (Altschul et al, J. Mol. Biol. 215: 403-410, 1990). When using BLASTX to analyze amino acid sequence identity, the parameters are, for example, a score of 50 and a word length of 3. When using the BLAST and Gapped BLAST programs, the default parameters for each program are used. Specific methodology for these analysis methods is well known (http://www.ncbi.nlm.nih.gov).

The above LIV1-like proteins may be prepared by other known methods. For example, LIV1 DNA may be artificially altered using site-directed mutagenesis, such as methods for constructing deletion mutants using exonuclease, or cassette mutagenesis; the altered LIV1 DNA may then be used to prepare a desired protein.

As described in the Examples, the activity of proteins prepared as LIV1-like proteins as regulatory agents of Snail activity may be determined by using their activity in suppressing the expression of the cell adhesion molecule E-cadherin as an indicator, since Snail protein suppresses the expression of E-cadherin.

Another preferable example of substances available as regulatory agents of Snail activity is isolated DNAs comprising the sequence of SEQ ID NO: 3, 4, 25, 27, 29, 31, 33, 35, 37, 39, or 41. The above DNAS encode LIV1 protein, and once introduced into a cell, they can indirectly regulate Snail activity through LIV1 protein expression.

The above DNAs may be prepared from cDNAs from LIV1-expressing cells by hybridization techniques well known to those skilled in the art, using a part of the sequence of SEQ ID NO: 3, 4, 25, 27, 29, 31, 33, 35, 37, 39, or41 as a probe. In addition, the DNAs may be obtained from mRNAs by performing RT-PCR using a part of the sequence of SEQ ID NO: 3, 4, 25, 27, 29, 31, 33, 35, 37, 39, or 41 as a primer. Alternatively, such DNAs may be artificially synthesized using a commercially available DNA synthesizer.

Furthermore, DNAs similar to the above DNAs are another example of regulatory agents of Snail activity. Such DNAs include DNAs that hybridize under stringent conditions with the sequence of SEQ ID NO: 3, 4, 25, 27, 29, 31, 33, 35, 37, 39, or 41, and that encode a protein capable of regulating Snail activity. Methods for preparing such DNAs are as described above.

Each of the above-described DNAs may be inserted into appropriate vectors for use. Such DNAs inserted into vectors is another embodiment of the regulatory agents of Snail activity. The vectors for use may be expression vectors appropriately selected depending on the purpose. Specifically, mammalian-derived vectors (such as pcDNA3 (Invitrogen Inc.), pEGF-BOS (Nucleic Acids Res., 18(17), p.5322, 1990), pEF, pCDM8, and pCXN), insect cell-derived vectors (such as the “Bac-to-Bac baculovirus expression system” (Invitrogen Inc.) and pBacPAK8), plant-derived expression vectors (such as pMHl and pMH2), animal virus vectors (such as pHSV, pMV, and pAdexLcw), retroviral vectors (such as pZIPneo), yeast-derived vectors (such as “Pichia Expression Kit” (Invitrogen Inc.), pNV11, and SP-Q01), Bacillus Subtilis-derived vectors (such as pPL608 and pKTH50), E. coli vectors (such as the M13 vector series, pUC vector series, pBR322, pBluescript, and pCR-Script) and such may be used. Herein, the use of vectors that can be expressed in mammalian cells is preferable, and the use of expression vectors is preferable. Vectors maybe introduced into cells by methods selected from, for example, calcium phosphate methods (Virology, Vol.52, p.456, 1973), DEAE-dextran method, methods using cationic liposome DOTAP (Roche Diagnostics), electroporation methods (Nucleic Acids Res., Vol 15, p.1311, 1987), lipofection methods (J. Clin. Biochem. Nutr., Vol.7, p.175 1989), transfection mediated by viral infection (Sci. Am., p.34, 1994), and particle guns.

As described above, a variety of regulatory agents of Snail activity directly or indirectly induce activation of the zinc finger protein Snail. Furthermore, Snail activation contributes to EMT induction, and EMT relates to gastrulation in embryos, regeneration of organs and tissues, and cancer metastasis and progression. Thus, the regulatory agents of Snail activity may be used to elucidate the developmental processes and the mechanisms of cancer metastasis and progression. Furthermore, such regulatory agents may be effectively used as agents for promoting organ- and tissue-regeneration in regenerative medicine. In addition, because the regulatory agents promote cell regeneration, they may be applied as wound-healing agents.

The present invention relates to agents for suppressing Snail activity. Suppression of Snail activity inhibits Snail's suppression of adhesion molecule expression, which leads to inhibition of EMT induction and prevention of cancer metastasis and progression.

Examples of the agents for suppressing Snail activity may be antisense oligonucleotides targeting DNAs or mRNAs that encode LIV1. Because LIV1 can regulate Snail activity, as demonstrated by the present inventors, antisense oligonucleotides against DNAs encoding LIV1 are expected to inhibit expression of the endogenous LIV1 gene, and thereby negatively regulate Snail activity. Examples of such antisense oligonucleotides are antisense oligonucleotides that target DNAs comprising the sequence of SEQ ID NO: 3, 4, 25, 27, 29, 31, 33, 35, 37, 39, or 41, or mRNAs generated from these DNAs. As an example, oligonucleotides of SEQ ID NOs: 5 and 6 may be used. In addition, any oligonucleotides may be included in the antisense oligonucleotides of this invention, as long as they can hybridize with a part of a DNA comprising the sequence of SEQ ID NO: 3, 4, 25, 27, 29, 31, 33, 35, 37, 39, or 41, and effectively inhibit LIV1 expression. Such oligonucleotides need not be completely complementary to the DNAs or corresponding mRNAs comprising the nucleotide sequence of SEQ ID NO: 3, 4, 25, 27, 29, 31,33,35,37,39,or 41.

The above-described antisense oligonucleotides may be inserted in an appropriate vector for use, depending on the purpose. For example, vectors used in gene therapy applications may be appropriately selected from viral vectors such as retrovirus vectors, adenovirus vectors, and vaccinia virus vectors, non-viral vectors such as cationic liposomes and ligand-DNA complexes, and such. Further, antisense oligonucleotides can be administered as naked plasmid DNAs (naked pDNAs) along with a large volume of aqueous solution, without using a carrier.

Another example of the agents for suppressing Snail activity may be double-stranded RNAs comprising a sequence identical or similar to a part of a DNA encoding LIV1. Such double-stranded RNAs comprising a sequence identical or similar to a target gene sequence may trigger RNA interference (RNAi), which inhibits expression of the target gene. RNAi is a phenomenon caused when a double-stranded RNA (dsRNA) is introduced into cells, specifically degrading the cellular mRNA that corresponds to the RNA sequence and preventing its expression as a protein. The regions forming double strands may form double strands over an entire region, or parts (both or either of the termini, for example) may form single strands. Thus, the double-stranded RNAs of this invention may also comprise regions that are not double stranded. The oligo RNAs used for RNAi are often 10-100 bp, and normally 19-23 bp. RNAi may be performed according to the methods described in Nature, Vol.391, p.806, 1998;
Proc.Natl.Acsd.Sci.USA, Vol.95, p.15502, 1998; Nature, Vol.395, p.854, 1998;
Proc.Natl.Acsd.Sci.USA, Vol.96, p.5049, 1999; Cell, Vol.95, p.1017,1998;
Proc.Natl.Acsd.Sci.USA, Vol.96, p.1451, 1999; Proc.Natl.Acsd.Sci.USA, Vol.95, p.13959, 1998;
Nature Cell Biol., Vol.2, p.70, 2000, etc.

The above-described agents for suppressing Snail activity may be used for studying Snail-related embryology and oncology, and in addition, they can conceivably be used as therapeutic pharmaceuticals for suppressing cancer metastasis and progression by the negative regulation of Snail activity. They are expected to be effective for cancers in tissues where LIV1 is expressed, and especially effective for breast cancers. In addition, since they can suppress cell adhesion and cell movement, they are expected to suppress the invasion and spread of inflammatory cells, and can therefore be applied as anti-inflammatory agents.

Gene therapy is an example of the methods for using the above agents for suppressing Snail activity for cancer therapy. Gene therapy is a method for treating a disease by introducing an exogenous normal gene into patient cells and changing the cellular phenotype to correct a mutated gene. Gene therapy is thought to be effective for treating not only genetic diseases but also other diseases, such as AIDS and cancers. Gene therapy is classified into: methods where a gene is directly introduced in vivo to be incorporated into cells (in vivo methods); and methods where patient cells are sampled, a gene is introduced thereto ex vivo, and then the cells are transplanted back into the patient (ex vivo methods). In in vivo gene therapy methods, the above agents for suppressing Snail activity may be introduced in vivo by an administration method such as intramuscular injection, local injection, rubbing, and inhalation, either directly or through insertion into a vector.

The proteins, DNAs and oligonucleotides of this invention may be used as pharmaceuticals by directly administrating these substances, or preparing formulations using known formulation techniques. For example, they may be formulated in combination with a pharmacologically acceptable vehicle or stabilizer. The administration route, dose, and methods may be appropriately selected according to the purpose and subject of treatment.

The present invention also provides EMT-inducing agents. As described above, LIV1 activates Snail, which induces EMT. Thus, the above-described regulatory agents of Snail activity may also be used as EMT regulatory agents.

Herein, the term “EMT-inducing agents” refers to substances capable of directly or indirectly inducing epithelial-mesenchymal transition (EMT).

Examples of the EMT-inducing agents may be isolated LIV1 proteins comprising the amino acid sequence of SEQ ID NO: 1, 2, 26, 28, 30, 32, 34, 36, 38, 40, or 42, and proteins similar to LIV1.

Methods for preparing LIV1 proteins are as described above.

The above-described proteins similar to LIV1 are those capable of inducing EMT. Examples of the proteins may be proteins comprising an amino acid sequence with one or more amino acid substitutions, deletions, insertions, and/or additions in the amino acid sequence of SEQ ID NO: 1, 2, 26, 28, 30, 32, 34, 36, 38, 40, or 42, and proteins encoded by DNAs that hybridize under stringent conditions with a DNA comprising the nucleotide sequence of SEQ ID NO: 3, 4, 25, 27, 29, 31, 33, 35, 37, 39, or 41.

Preparation methods are the same as those described in the section on the regulatory agents of Snail activity. To confirm the activity of an EMT-inducing agent, as described in the Examples, a test substance may be injected into an embryo in which LIV1 is specifically suppressed, and cell migratory movements and morphological changes can be observed through the test substance's effects. If the observed results of cell migratory movements and morphological changes are the same as those in an embryo where LIV1 is not specifically suppressed, the test substance is considered to have an effect as an EMT-inducing agent.

Another preferable example of a substance useful as an EMT-inducing agent may be an isolated DNA comprising the sequence of SEQ ID NO: 3, 4, 25, 27, 29, 31, 33, 35, 37, 39, or 41. The above-described DNAs encode a LIV1 protein, and once introduced into cells they can indirectly induce EMT through LIV1 protein expression. Furthermore, DNAs similar to the above DNAs and vectors into which the DNAs are inserted can be used as EMT-inducing agents. Methods for preparing them are described above.

Since EMT is involved in the gastrulation of embryos, the regeneration of organs and tissues, and cancer metastasis and progression, these EMT-inducing agents may be used for elucidating the developmental processes and mechanisms of cancer metastasis and progression. Moreover, by promoting cellular regeneration, these agents may be effectively used as agents for promoting organ- and tissue-regeneration in regenerative medicine, and as wound-healing agents.

The present invention relates to EMT-suppressing agents. Suppression of EMT leads to prevention of cancer metastasis and progression by inhibiting EMT induction. For example, the EMT-suppressing agents of this invention are useful for suppressing estrogen receptor-negative (ER(−)) breast cancers and their metastasis.

Since the present inventors revealed that LIV1 can induce EMT, antisense oligonucleotides targeting DNA or MRNA sequences encoding LIV1 are thought to interfere with the expression of the endogenous LIV1 gene, thereby suppressing EMT. Such antisense oligonucleotides may include those targeting DNAs comprising the sequence of SEQ ID NO: 3, 4, 25, 27, 29, 31, 33, 35, 37, 39, or 41, or mRNAs purified from these DNAs. For example, oligonucleotides of SEQ ID NOs: 5 and 6 may be used. In addition, the antisense oligonucleotides of this invention include any oligonucleotides as long as they can hybridize with a part of a DNA comprising the nucleotide sequence of SEQ ID NO: 3, 4, 25, 27, 29, 31, 33, 35, 37, 39, or 41, or the like, and effectively inhibit the expression of LIV1. Such antisense oligonucleotides need not be completely complementary to the above DNAs comprising the sequence of SEQ ID NO: 3, 4, 25, 27, 29, 31, 33, 35, 37, 39, or 41, and the like.

Moreover, double-stranded RNAs comprising a sequence identical or similar to a part of a DNA encoding LIV1 are also examples of EMT-suppressing agents.

The embodiments for the use of LIV1-suppressing agents are the same as for the agents for suppressing Snail activity.

The present invention relates to methods for screening candidate substances for agents for suppressing Snail activity. The screening methods of this invention comprise the steps of: injecting one-cell stage embryos with a test substance and a vector which comprises a reporter gene operably linked under the control of the E-cadherin promoter; measuring the expression level of the reporter gene; and selecting a compound that reduces or increases the measured expression level of the reporter gene, compared with that in the absence of the test substance.

The reporter genes used in this invention are not limited as long as their expression is detectable. For example, luciferase, CAT gene, and β-galactosidase may be used.

Examples of test substances for the screening methods of this invention include expression products of gene libraries, synthetic low-molecular-weight compounds, synthetic peptides, natural compounds, and such.

Further, the present invention relates to regulatory agents of protein activities that require zinc (Zn). As described above, LIV1 protein is a zinc transporter, and is thought to regulate not only the activity of Snail, but also that of other proteins that require Zn. Thus, LIV1 is expected to be useful as a regulatory agent of protein activities that require Zn The regulatory agents of protein activities that require zinc of the present invention may be useful in studies for elucidating the molecular mechanisms to which proteins that requires Zn are related, and they may be applied as immune regulatory agents.

All prior art literatures cited in this description are incorporated herein by reference.

### EXAMPLES

### Example 1

### Identification of STAT3 Target Genes

To elucidate the molecular mechanism of STAT3, which is involved in gastrulation, downstream target genes of STAT3 were identified. First, to isolate STAT3-induced genes induced in the gastrula organizer, subtraction screening was performed using normal embryos and STAT3-morphant embryos.

The term “morphant embryo” refers to an embryo in which the expression of a particular gene is fully inhibited by morpholino. In this Example, morphant embryos with inhibited expression of STAT3 gene were generated. To generate the morphant embryos, two kinds of STAT3-morpholino nucleotides (STAT3-MO) were prepared. STAT3D4-MO were also prepared as controls, by adding a mutation to the STAT3-MO. These morpholino oligonucleotides were generated by GENE TOOLS. The sequences of the above morpholino oligonucleotides are shown below:
STAT3-MO:

(SEQ ID NO: 9)

5′-CTCAAGGTTTCAGATAAATCGTCCT-3′

(SEQ ID NO: 10)

5′-GCCATGTTGACCCCTTAATGTGTCG-3′

STAT3D4-MO:

(SEQ ID NO: 11)

5′-CTCtAGGaTTCAGATAAAaCGTgCT-3′

(SEQ ID NO: 12)

5′-GCCtTGTaGACCCCTTAAaGTGaCG-3′

The two kinds of STAT3-MO were injected into the yolk of one-cell stage embryos in equal amounts to prepare the morpholino embryos (STAT3-MO-injected). The control embryos (STAT3D4-MO-injected) were similarly generated.

Poly (A)+RNAs were isolated from the STAT3-MO-injected mid-gastrula-stage embryos and STAT3D4-MO-injected mid-gastrula-stage embryos. Subtraction screening was performed using PCR-Select™ cDNA subtraction kits (Clontech), as per to the manufacturer's protocol.

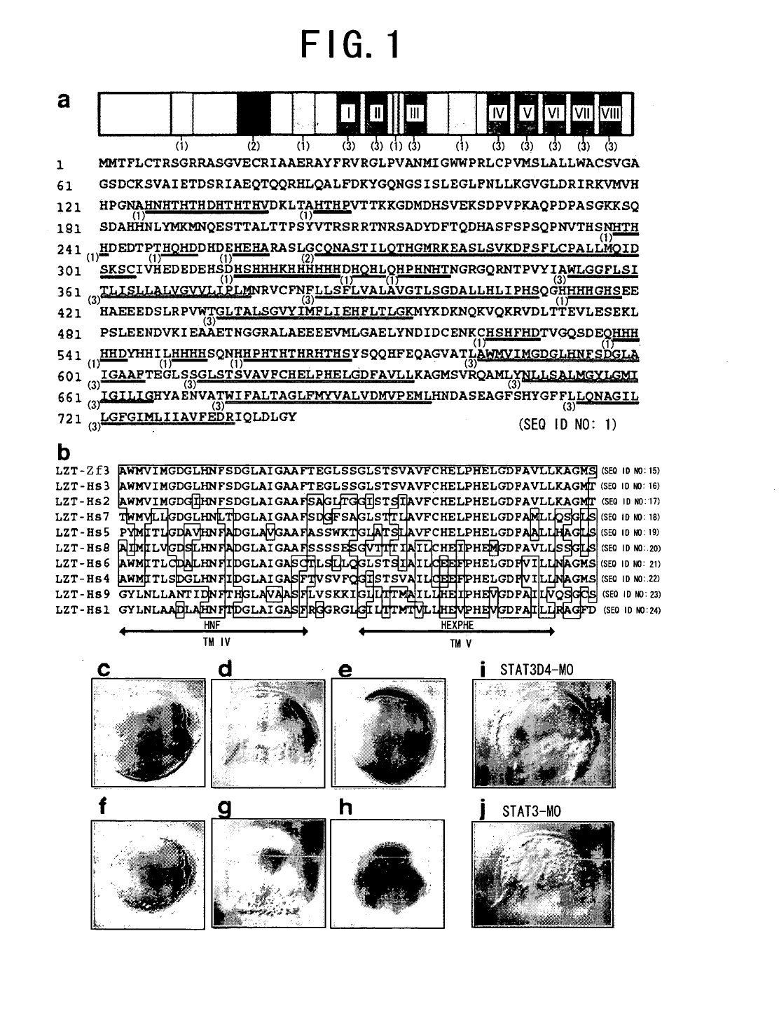

The sequences of the cDNAs isolated by the above subtraction screening were determined using standard methods, and subjected to database searches. Unexpectedly, one of the cDNAs isolated by the present inventors turned out to be zebrafish LIV1 (LZT-Zf3; SEQ ID NO: 3). The deduced amino acid sequence of LIV1 and the LIV1 protein domains are shown in SEQ ID NO: 1 and FIG. 1a. Since the sequence comprises a long extracellular N-terminus, a short extracellular C-terminus, a long variable region in the cytoplasmic loop between the TMIII and TMIV domains, an HNF motif in the TMIV domain, an HEXPHE motif in the TMV domain, and eight transmembrane (TM) domains comprising extensive histidine-rich repeats, it was characterized as the ZIP family of zinc transporters (Taylor K. M., and Nicholson R. I. The LZT proteins; the LIV-1 subfamily of zinc transporters. Biochim. Biophys. Acta 1611: 16-30 (2003)). The above cDNAs were identified as zebrafish homologues of human LIV1 by phylogenic tree analysis and alignment of the regions spanning the transmembrane domains IV and V of the LZT subfamily .of ZIP transporters (LZT-Hs3; FIG. 1b). The present inventors had previously demonstrated that STAT3 is activated in prechordal mesendodermal cells (Yamashita S. et al. Stat3 Controls Cell Movements during Zebrafish Gastrulation. Dev Cell 2, 363-75 (2002)).

Based on the above findings, the expression of LIV1 mRNA in zebrafish embryos at the gastrula stage was examined using the WISH method.

The whole-mount in situ hybridization (WISH) method was basically performed using the zebrafish LIV1 CDNA sequence (SEQ ID NO: 1) as a probe for LIV1 MRNA, according to previously described methods (Yamashita S. et al. Stat3 Controls Cell Movements during Zebrafish Gastrulation. Dev Cell 2, 363-75 (2002)).

The results showed that zebrafish LIV I mRNA was maternally expressed, and that embryonic transcripts increased at the shield stage and accumulated in the prechordal mesendodermal cells in which STAT3 was activated (FIGS. 1c-h). Expression of LIV1 mRNA in the gastrula organizer was completely absent from STAT3-morphant embryos, indicating that LIV1 is a downstream target of STAT3 (FIGS. 1i and j).

### Example 2

### Examination Of Liv1 Function In Early Stage Embryos

To examine the role of LIV1 in zebrafish embryos in the early stages of gastrulation, LIV1 antisense morpholino (LIV1-MO), a specific translation inhibitor (Non-Patent Document 21: Nasevicius A. and Ekker, S. C. Effective targeted gene ‘knockdown’ in zebrafish. Nat Genet 26, 216-20 (2000)), was used to generate embryos lacking in LIV1 activity, and the effect of losing LIV1 activity was observed. The LIV1-MO and control oligonucleotide sequences are shown below:
LIV1-MO:

(SEQ ID NO: 5)

5′-CGGAAACAGCGCGAGTGTCTTTTGT-3′

(SEQ ID NO: 6)

5′-ACCGTGTGCAAAGAAACGTCATCAT-3′

LIV1D4-MO (a control in which the sequence of LIV1-antisense morpholin was mutated):

(SEQ ID NO: 7)

5′-CGcAAAaAGCGaGAGTGTaTTaTGT-3′

(SEQ ID NO: 8)

5′-AcgGTcTGCAAAcAAACGTgATgAT-3′

Morpholino embryos were generated by using these morpholino oligonucleotides as described for the above STAT3-morpholino embryos.

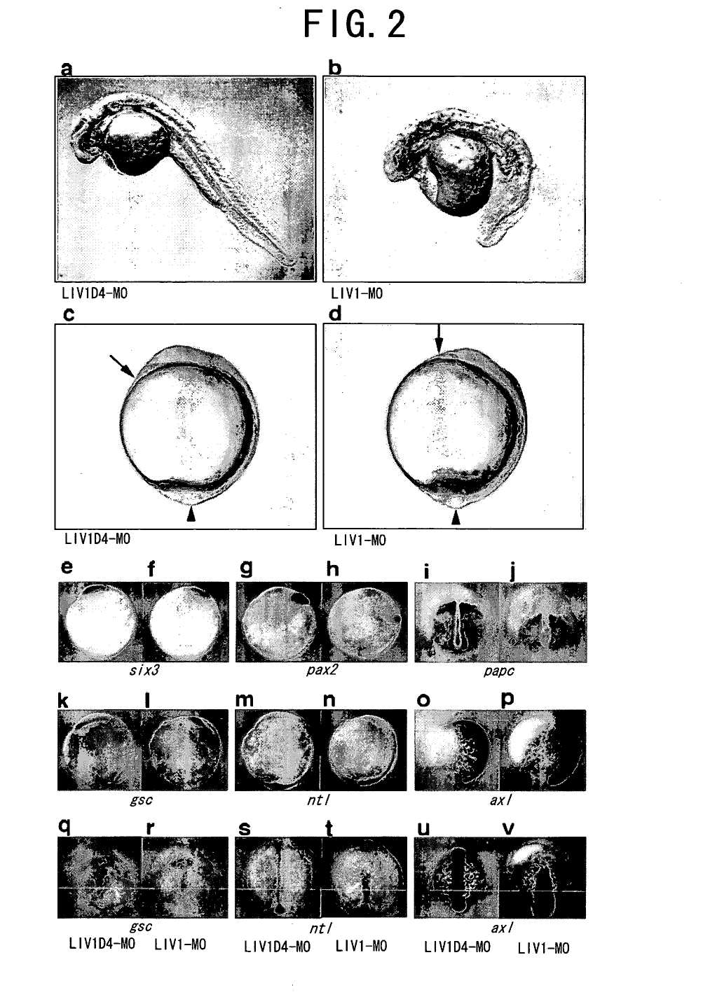

Embryos injected with LIV1-MO were confirmed to have mislocated heads and shortened anterior-posterior axis in the later and final stages of gastrulation. On the other hand, embryos injected with the control LIV1D4-MO did not show any obvious phenotypic changes (FIGS. 2a-d). In the LIV1-MO-injected zebrafish embryos, the axial anterior-most mesendodermal (Polster) and anterior ectodermal structures could be observed with the naked eye, and were transposed toward the vegetal pole, up to 45 degrees from the normal position at the animal pole. Moreover, although the axial hypoblast (notochord and somite) was formed in the LIV1-MO-injected zebrafish embryos, it was shorter and thicker than normal. This suggests that the anterior movement of the axial mesendoderm was severely impaired, whereas the involution, epiboly, and dorsal convergence movements were normal. Consistent with this, at the shield stage the LIV1-MO-injected embryos showed a germ ring (a hypoblast of mesendoderm formed by the involution movement of surrounding cells) and embryonic shield (a thickening of the dorsal surroundings; gastrula organizer) (data not shown). Taken together, these results indicate that during the gastrulation process the LIV1-depleted-zebrafish-embryos retained the ability to form mesendoderm, organizer, and dorsal-ventral and anterior-posterior axes, and the ability to initiate involution, epiboly, and dorsal convergence movements. However, axial mesendodermal cells in the LIV1-MO-injected embryos did not move anteriorly, and head positioning and elongation of the anterior-posterior axis were impaired.

Next, marker gene expression was confirmed using the WISH method. Six3, pax2, goosecoid, no tail, axial, and papc were used as the markers.

The results also provided evidence for morphological abnormalities associated with the loss ofLIVl function (FIGS. 2e-v). The markers for the forebrain (six3), mid-hindbrain bundle (pax2), anterior axial mesoderm (goosecoid), posterior axial mesoderm (no tail and axial), paraxial mesoderm (papc), and endoderm (axial) were expressed in the LIV1-MO-injected embryos at the late gastrula stage, but the expression domains of these marker genes were mislocated on the vegetal pole. In addition, shortening of the expression domain in the anterior-posterior axis, and slight mediolateral expansion were confirmed in the LIV1-MO-injected embryos. Consistent with normal fate specification and regionalization in LIV1-deficient embryos at the late gastrula stages, normal expression of the above mesendodermal genes was observed at the earlier stages of development (data not shown). These results indicate that LIV1 plays an essential role in the gastrulation process by affecting cell movement, without significantly altering the early cell-fate specification of mesendodermal cells.

### Example 3

### Examination Of LIV1 Function During Gastrulation

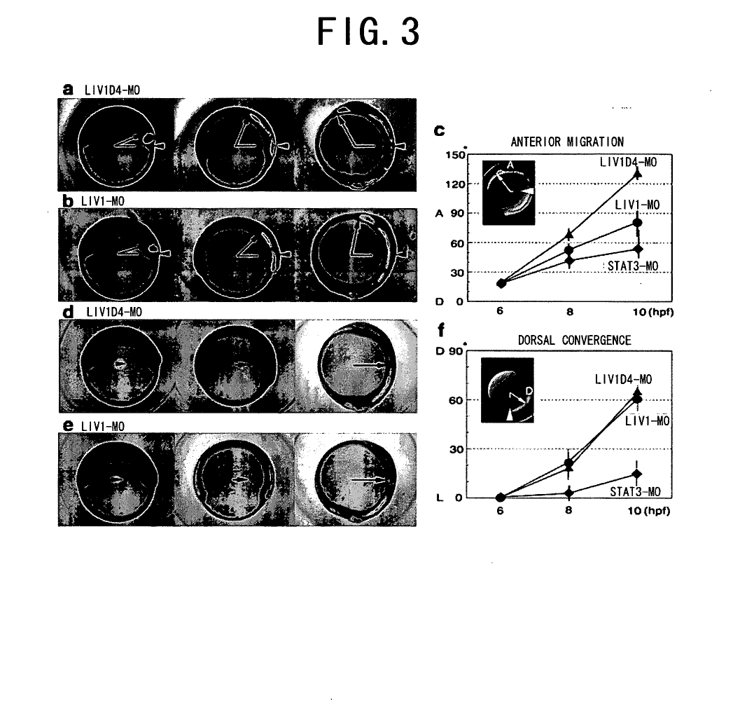

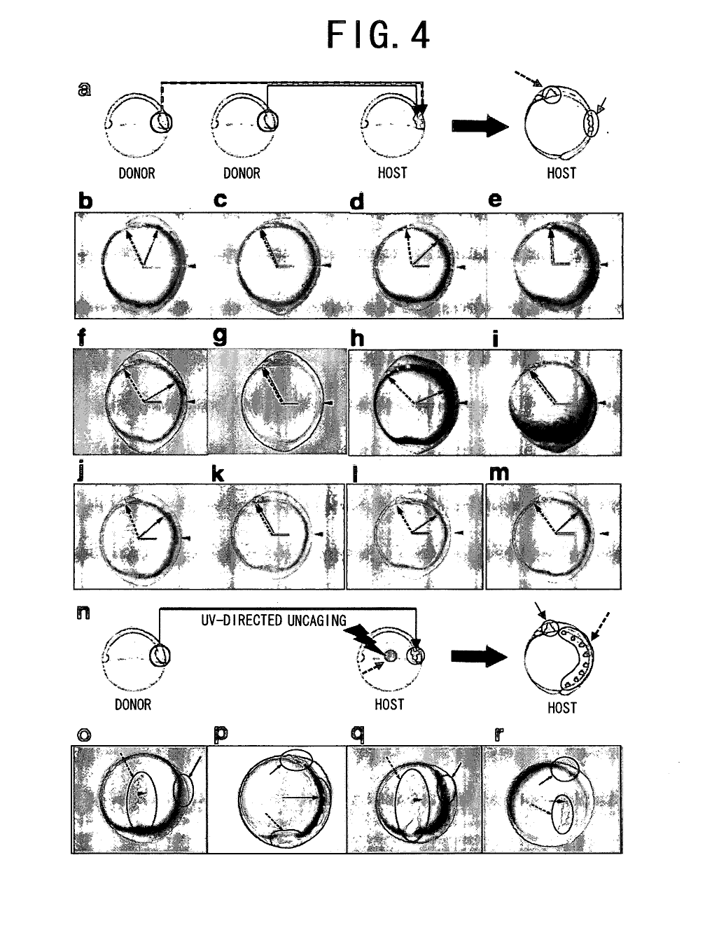

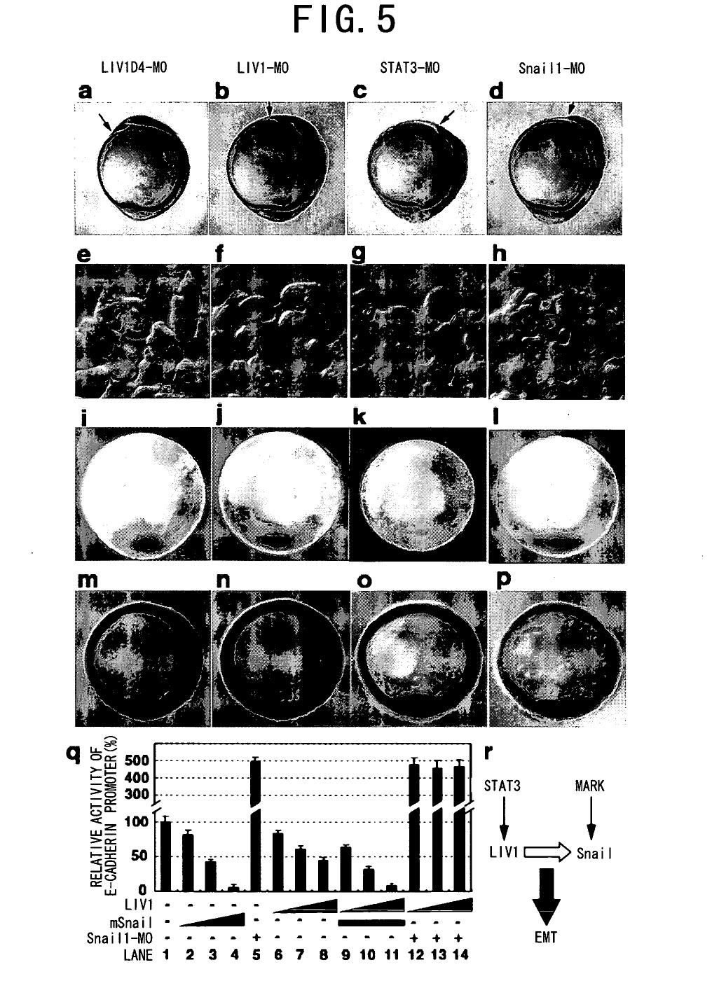

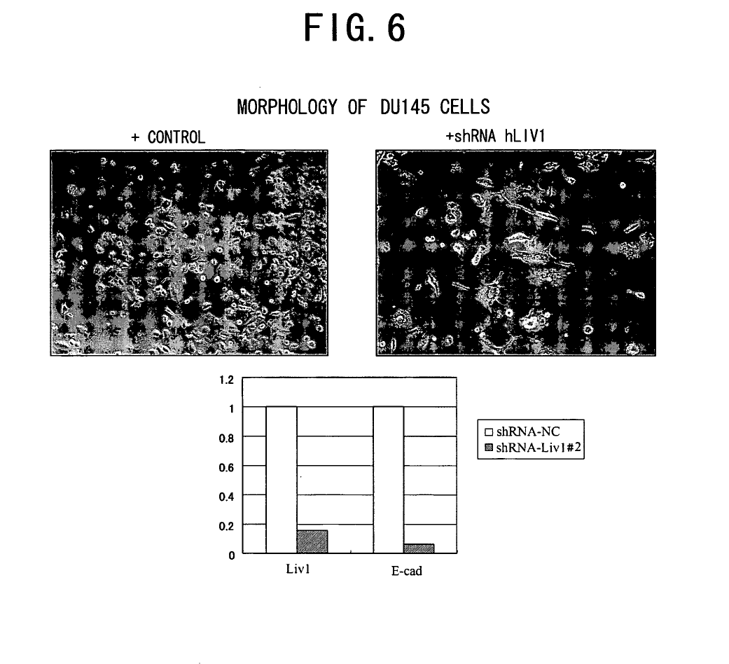

To further examine the effect of LIV1 on cell movement during gastrulation, cell-tracing experiments were performed in zebrafish embryos using 4,5-dimethoxy-2-nitrobenzyl (DMNB)-caged fluorescein dextran (Kozlowski, D. J. and Weinberg, E. S. Photoactivatable (caged) fluorescein as a cell tracer for fate mapping in the zebrafish embryo. Methods Mol Biol 135, 349-55 (2000)) (FIGS. 3a-f).

Cell-tracing experiments were performed essentially according to previously described methods (Yamashita, S. et al. Stat3 Controls Cell Movements during Zebrafish Gastrulation. Dev Cell 2, 363-75 (2002)). Specifically, 100 pl of 0.5% DMNB-caged fluorescein-dextran (molecular weight 10,000; Molecular Probes) was injected into the yolk cell of one-cell-stage embryos previously injected with 10 ng of LIV1D4-MO or LIV1-MO. To uncage at the shield stage, a beam of ultraviolet light (λ<360 nm), generated using a DAPI filter set, was directed for one second at the dorsal or lateral blastoderm margin. Then, the cells in the embryonic shield were labeled, and the position of the labeled cells during gastrulation was traced.

At the end of gastrulation the labeled cells in the control LIV1D4-MO-injected embryos (FIG. 3a) were distributed in the dorsal axial hypoblast along the entire length of the AP axis structure, as reported previously (Yamashita, S. et al. Stat3 Controls Cell Movements during Zebrafish Gastrulation. Dev Cell 2, 363-75 (2002)). In contrast, the anterior movement of the labeled cells in the LIV1-MO-injected embryos (FIG. 3b) was disturbed, resulting in a shortened axial mesendoderm. To monitor the dorsal convergence movements, cells in the lateral blastoderm margin, 90 degrees from the dorsal embryonic shield, were labeled at the shield stage. During gastrulation, the labeled cells in the LIV1-MO-injected embryos moved dorsally and anteriorly, and just as for the control LIV1D4-MO-injected embryos, at the end of gastrulation they extended along the anterior-posterior-axis structure (FIGS. 3d and e). These results clearly show that LIV1 is essential for the active migration of organizer cells, but not for the dorsal convergence of non-axial mesodermal cells.

The above observations suggest that LIV1 is one of the essential STAT3 target genes, and that LIV1 is required for STAT3's cell-autonomous roles, but not for its non-cell-autonomous roles. If this is so, LIV1 is predicted to act cell-autonomously in the active migration of organizer cells.

Transplantation experiments were performed to confirm the above prediction. The transplantation experiments were performed according to previously described methods (Yamashita, S. et al. Stat3 Controls Cell Movements during Zebrafish Gastrulation. Dev Cell 2, 363-75 (2002)). Specifically, donor embryos were prepared by injecting the yolk cell of one-cell-stage embryos with 100 pl of 0.5% rhodamine-dextran (molecular weight 10,000; Molecular Probes) or 100 pl of 0.5% fluorescein-dextran (molecular weight 10,000; Molecular Probes), with 10 ng of predetermined morpholino. A small population of deep cells (10 to 30 cells) obtained from the embryonic shield of donor embryos was transplanted into the embryonic shield of host embryos at the same developmental stage. In addition, the plasmid pCS2+zebrafish LIV1 (NotI, SP6) was linearized to prepare sense-strand capped mRNA, which was used for injection into cells.

First, prechordal mesendodermal cells, obtained from the embryonic shield of LIV1-MO-injected donor embryos or LIV1D4-MO-injected control donor embryos, were co-transplanted into the embryonic shield of normal host embryos or LIV1-depleted embryos, and the cells were traced (FIGS. 4a-e). When the transplanted prechordal mesendodermal cells were derived from control donors, and not from LIV1-depleted donors, the cells migrated anteriorly to the animal pole in both the normal host embryos (FIG. 4b) and the LIV1-depleted host embryos (FIG. 4d). The anterior migration of the LIV1-MO-injected cells was reduced, however, in both the normal control hosts (FIG. 4c) and the LIV1-depleted hosts (FIG. 4e), it was rescued by co-injecting LIV1 mRNA into the LIV1-depleted donor cells. These results indicate that with regards to the anterior migration of the prechordal mesendoderm, LIV1 acts on cells autonomously.

### Example 4

### Examination of the Involvement of LIV1 in the Role of STAT3

Next, to examine whether LIVl can sufficiently restore those functions impaired by STAT3 depletion in the anterior migration of STAT3-depleted prechordal mesendodermal cells, transplantation and cell-tracing experiments were performed. STAT3 MRNA for injection was prepared by linearizing pCS2+zebrafish STAT3 (NotI, SP6) (Yamashita, S. et al Stat3 Controls Cell Movements during Zebrafish Gastrulation. Dev Cell 2, 363-75 (2002)).

As shown in FIG. 4f, when STAT3-depleted cells were transplanted into the embryonic shield, the cells did not migrate anteriorly beyond the animal pole. This dysfunction was rescued by the co-injection of STAT3 mRNA (FIG. 4g), but not of STAT5 mRNA (FIG. 4h). The most significant of the results obtained in this Example is that LIV1 mRNA rescued the defects of STAT3-depleted cells (FIG. 4i). These results clearly show that LIV1 sufficiently rescues the anterior migration function of prechordal mesendodermal cells in STAT3-depleted embryos.

The constraints of LIV1, predicted from the above observations, were further clarified from the results of the cell-tracing experiments below, showing that LIV1 mRNA could not rescue dysfunctions in the non-cell-autonomous role of STAT3 in shield cells, that is, dysfunctions in the induction of convergence.

Prechordal mesendodermal cells obtained from donors injected with STAT3-morpholino and predetermined mRNAs were transplanted into the shield region, and the lateral mesendodermal cells in hosts co-injected with STAT3-morpholino and caged FITC were then labeled by UV-directed uncaging at the early gastrula stage. As shown in FIGS. 4n-r, reduced convergence observed in the STAT3-depleted host embryos was rescued by the transplantation of prechordal mesendodermal cells from STAT3-depleted donors injected with STAT3 mRNA (FIG. 4p), but not by those cells from STAT3-depleted donors injected with LIV1 mRNA (FIG. 4r).

All these data established that LIV1 is an essential and sufficient target gene for the cell-autonomous roles of STAT3, but not for its non-cell-autonomous roles. LIV1 is suggested to play a key role in the EMT of organizer cells regulated by STAT3 activity.

### Example 5

### Examination of the Relationship between the LIV1-Induced EMT of Organizer Cells and Snail

As described above, LIV1 has been shown to induce the EMT of gastrula organizer cells. Accordingly, the present inventors examined whether LIV1 affects the expression and/or activity of Snail during induction of organizer cell EMT by LIV1.

First, to examine the effect on Snail activity, the phenotypes at the end of gastrulation of LIV1-depleted, STAT3-depleted, and Snail-depleted embryos, and the migratory behavior of organizer cells in the embryos, were analyzed (FIGS. 5a-h). LIV1-depleted embryos and STAT3-depleted embryos were prepared as described above. The Snail-MO sequence used for preparing Snail-depleted embryos is shown below:

(SEQ ID NO: 13)

5′-GTCCACTCCAGTTACTTTCAGGGAT-3′

(SEQ ID NO: 14)

5′-CATGCTGAACTCTGAAGTTGATC-3′

During the gastrulation process, the organizer cells in normal embryos actively weakened the cell-cell adhesion system, and left their marginal region to migrate individually, resulting in full anterior extension along the body axis at the end of gastrulation (FIGS. 5a and e). However, in LIV1-depleted embryos, the organizer cells could not weaken their association, resulting in severe disturbance of organizer cell migration, mislocation of the head, and the formation of a shortened anterior-posterior axis structure (FIGS. 5b and f). Similar defects in the migratory behavior of organizer cells and body axis extension were also observed in STAT3-depleted embryos (FIGS. 5c and g) and Snail-depleted embryos (FIGS. 5d and h). These abnormal organizer cell behaviors clearly indicated a severe disturbance of organizer cell EMT in LIV1-depleted embryos, as in Snail-depleted embryos.

Next, Snail expression was examined. Snail1 mRNA used for injection was prepared by linearizing pCS2+mouse Snail (NotI, SP6) (Yamashita, S. et al. Stat3 Controls Cell Movements during Zebrafish Gastrulation. Dev Cell 2, 363-75 (2002)). LIV1 mRNA and Snail1 mRNA were normally expressed in Snail 1-MO-injected embryos and LIV1-MO-injected embryos respectively (FIGS. 5i-p).

These results indicate that LIV1 and Snail1 are essential for EMT during the gastrulation process, and that the expression of LIV1 and Snail1 are independently regulated in gastrula organizer cells. On the other hand, cell-autonomous defects in the anterior migration of Snaill-depleted organizer cells were not rescued by co-injection of LIV1 mRNA (FIG. 4l), and vice versa (data not shown). These results suggested that LIV1 may affect Snail activity.

To address the above issue, the effect of LIV1 on the repressor activity of Snail was examined by reporter assay using a Snail-responsive reporter plasmid (Batlle, E. et al. The transcription factor snail is a repressor of E-cadherin gene expression in epithelial tumor cells. Nat Cell Biol 2, 84-9 (2000)) (FIG. 5q).

The reporter assay was essentially performed according to previously described methods (Batlle, E. et al. The transcription factor snail is a repressor of E-cadherin gene expression in epithelial tumor cells. Nat Cell Biol 2, 84-9 (2000)). Specifically, a reporter construct pGL3-E-cadh promoter (2.5 pg/embryo), the pGL3 vector comprising the Luc gene under the control of the human E-cadherin promoter (-178 to +92), and a control reporter vector (pRLtk) (0.5 g/embryo) were injected into one-cell-stage embryos with or without zebrafish LIV1 RNA (1, 10, or 100 pg/embryo), mouse Snail (1, 10, or 100 pg/embryo), or zebrafish Snail1 morpholino (10 pg/embryo). Firefly luciferase (Luc) and Renilla reniformis luciferase (RLluc) activities were measured using the Dual Luciferase Reporter Assay System (Promega) at the shield stage (6 hpf.), according to the manufacturer's protocol. Luciferase activity was always normalized using Rluc activity.

Snail RNA co-injected with the reporter plasmid into zebrafish embryos suppressed transcription from the reporter plasmid in a manner dependent on the dose of Snail RNA (FIG. 5q; lanes 1-4). In contrast, injection of Snail-MO into zebrafish embryos enhanced transcription from the reporter plasmid (FIG. 5q; lane 5). Similarly, LIV1 RNA also suppressed transcription from the reporter plasmid in a dose-dependent manner (FIG. 5q; lanes 6-8). Furthermore, co-injection of various amounts of LIV1 RNA with a constant small amount of Snail RNA enhanced the repressor activity of Snail in a manner dependent on the LIV1 RNA dose (FIG. 5q; lanes 9-11).

As LIV1 is a zinc transporter protein, LIV1 is very likely to regulate the activity of the zinc finger protein Snail. If this is so, reporter suppression by LIV1 is thought to be sensitive to Snail-specific translation inhibitors. As shown in FIG. 5q, LIV1 activity was completely absent in Snail1-MO-injected zebrafish embryos (FIG. 5q; lanes 12-14). In the cell-tracing experiments, the cell-autonomous rescue by LIV1 of the anterior migration defects of STAT3-depleted organizer cells was also sensitive to Snail-MO (FIGS. 4i and m). These results, along with the results of phenotypic and gene expression analyses (FIGS. 5a-p), provide evidence that LIV1 activates Snail and induces the EMT of organizer cells during gastrulation in zebrafish.

### Example 6

### Analysis of the LIV1 Function in DU145 (Human Prostate Cancer Cells)

To clarify the function of LIV1 in human cells, LIV1 function was analyzed in DU145 (human prostate cancer cells). The endogenous human LIV1 in DU145 was silenced by RNAi, and changes in cell morphology were observed.

First, DU145 cells were transfected with an hLIV1-targeting shRNA (AGGAGAAAGTAGATACAGA; SEQ ID NO: 43). Transfection was performed using the Lipofectamine method (Invitrogen) in the presence of 10% fetal calf serum (FCS) for ten hours. The piGENE PUR hU6 (iGENE), which carries puromycin resistant marker, was used as a vector for the transfection. As a control experiment, a control shRNA was transfected into other DU145 cells.

Next, DU145 cells into which LIV1 shRNA or control shRNA were respectively introduced, were cultured in the presence of 2 μg/ml of puromycin dihydrochloride (Puromycin Dihydrochloride from Streptomyces alboniger; Nakarai standard, Special Grade) in 10% FCS-DMEM medium for about a week.

In DU145 cells in which human LIV1 was silenced, morphological observation of each of the cells cultured for about a week confirmed morphological changes in mesenchymal-like-cells with numerous projections (FIG. 6).

The expression level of LIV1 and E-cadherin in the cultured cells was confirmed by RT-PCR. The results showed that the expression of both LIV1 and E-cadherin was suppressed in DU145 cells in which human LIV1 was silenced by RNAi (FIG. 6).

Furthermore, forced expression of zebrafish LIV1 in DU145 cells was found to cause similar morphological changes in mesenchymal-like-cells.

### INDUSTRIAL APPLICABILITY

The present invention provides LIV1, which is a regulatory agent of Snail activity and an EMT-inducing agent. The present inventors demonstrated for the first time that LIV1, a downstream target of STAT3, plays a key role in the EMT of gastrula organizer cells by affecting Snail activity. Thus, the regulatory agents of Snail activity and EMT-inducing agents of the present invention may not only contribute to the advancement of developmental studies as substances which regulate development, but may also be applied in regenerative medicine as agents for promoting the regeneration of organs for transplantation. The present invention also provides agents for suppressing Snail activity and EMT-suppressing agents. Since EMT is deeply involved in cancer metastasis and progression, agents for suppressing Snail activity and EMT-suppressing agents, both of which suppress EMT, are expected to block cancer metastasis and progression, and may be applied as novel pharmaceuticals for treating cancer. The EMT-suppressing agents of the present invention are useful for suppressing estrogen receptor-negative (ER(−)) breast cancer and its metastasis.

1. A regulatory agent of Snail activity, which is an isolated DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

2. A regulatory agent of Snail activity, which is a vector into which a DNA of any one of the following (a) to (d) is inserted:
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

3. A regulatory agent of Snail activity, which is an isolated protein encoded by a DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

4. An agent for suppressing Snail activity, which is:
(a) an antisense oligonucleotide targeting a DNA sequence comprising the sequence of SEQ ID NO: 3, 4, 25, 27, 29, 31, 33, 35, 37, 39, or 41; or
(b) a double-stranded RNA comprising a sequence identical or similar to a portion of a DNA comprising the sequence of SEQ ID NO: 3, 4, 25, 27, 29, 31, 33, 35, 37, 39, or 41.

5. (canceled)

6. A pharmaceutical for treating cancer, which comprises a nucleotide or vector of any one of the following (a) to (c), as an active ingredient:
(a) an antisense oligonucleotide of a DNA comprising the sequence of SEQ ID NO: 3;
(b) a vector into which an antisense oligonucleotide of a DNA comprising the sequence of SEQ ID NO: 3 is inserted; and
(c) an oligonucleotide that is a double-stranded RNA comprising a sequence identical or similar to a portion of a DNA comprising the sequence of SEQ ID NO: 3.

7. A pharmaceutical for treating cancer, which comprises as an active ingredient, an agent for suppressing Snail activity, which is:
(a) an antisense oliponucleotide targeting a DNA sequence comprising the sequence of SEQ ID NO: 3, 4, 25, 27, 29, 31, 33, 35, 37, 39, or 41; or
(b) a double-stranded RNA comprising a sequence identical or similar to a portion of a DNA comprising the sequence of SEQ ID NO: 3, 4, 25, 27, 29, 31, 33, 35, 37, 39, or 41.

8 . An EMT-inducing agent, which is an isolated DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

9 . An EMT-inducing agent, which is a vector into which a DNA of any one of the following (a) to (d) is inserted:
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

10. An EMT-inducing agent, which is an isolated protein encoded by a DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41.

11. An EMT-suppressing agent, which is:
(a) an antisense oligonucleotide targeting a DNA sequence comprising the sequence of SEQ ID NO: 3, 4, 25, 27, 29, 31, 33, 35, 37, 39, or41 or
(b) a double-stranded RNA comprising a sequence identical or similar to a portion of a DNA comprising the sequence of SEQ ID NO: 3, 4, 25, 27, 29, 31, 33, 35, 37, 39, or 41.

12. (canceled)

13. A pharmaceutical for treating cancer, which comprises as an active ingredient, an EMT-suppressing agent, which is:
(a) an antisense oligonucleotide targeting a DNA sequence comprising the sequence of SEQ ID NO:3, 4, 25, 27, 29, 31, 33, 35, 37, 39, or 41; or
(b) a double-stranded RNA comprising a sequence identical or similar to a portion of a DNA comprising the sequence of SEQ ID NO: 3, 4, 25, 27, 29, 31, 33, 35, 37, 39, or 41.

14. A method of screening for a candidate substance for an agent for suppressing Snail activity, wherein the method comprises the following steps of (a) to (c):
(a) injecting a vector which comprises a reporter gene operably linked under the control of the E-cadherin promoter, and a test substance into a one-cell-stage embryo;
(b) measuring the expression level of the reporter gene; and
(c) selecting a compound that reduces or increases the measured expression level of the reporter gene, compared with the measured expression level in the absence of the test substance.

15. A wound healing agent, which comprises as an active ingredient:
(A) a regulatory agent of Snail activity, which is an isolated DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1.2,26,28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41; or
(B) a regulatory agent of Snail activity, which is an isolated protein encoded by a DNA of any one of the following (a) to (d):
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41; or
(C) an EMT-inducing agent, which is an isolated DNA of any one of the following (a) to
(a) a DNA encoding a protein comprising the amino acid sequence of SEQ ID NO: 1, 2, 26, 28, 30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1,2,26,28,30, 32, 34, 36, 38, 40, or 42; and
(d) a DNA hybridizing under stringent conditions with the sequence of SEQ ID NO: 3, 4, 25, 27, 29, 31, 33, 35, 37, 39, or 41 or
(D) an EMT-inducing agent, which is an isolated protein encoded by a DNA of any one of the following (a) to (d):
(a) a DNA encoding a protein comprising the amino acid sequence of SEQ ID NO: 1, 2, 26, 28,30, 32, 34, 36, 38, 40, or 42;
(b) a DNA comprising the sequence of SEQ ID NO: 3, 4, 25, 27, 29, 31, 33, 35, 37, 39, or 41;
(c) a DNA encoding a protein comprising an amino acid sequence with one or more amino acid substitutions, deletions, insertions, and/or additions in the amino acid sequence of SEQ ID NO: 1, 2, 26, 28, 30, 32, 34, 36, 38, 40, or 42; and
(d) a DNA hybridizing under stringent conditions with the sequence of SEQ ID NO: 3, 4, 25, 27, 29, 31, 33, 35, 37, 39 or 41.

16. An anti-inflammatory agent, which comprises, as an active ingredient, an agent for suppressing Snail activity, which is:
(a) an antisense oligonucleotide targeting a DNA sequence comprising the sequence of SEQ ID NO: 3,4,25,27,29, 31, 33, 35, 37, 39, or 41; or
(b) a double-stranded RNA comprising a sequence identical or similar to a portion of a DNA comprising the sequence of SEQ ID NO: 3, 4, 25, 27, 29, 31, 33, 35, 37, 39, or 41:
or an EMT-suppressing agent, which is:
a) an antisense oligonucleotide targeting a DNA sequence comprising the sequence of SEQ ID NO: 3, 4, 25, 27, 29, 31, 33, 35, 37, 39, or 41: or
b) a double-stranded RNA comprising a sequence identical or similar to a portion of a DNA comprising the sequence of SEQ ID NO: 3, 4, 25, 27, 29, 31, 33, 35, 37, 39, or 41.

17. A regulatory agent of a protein activity that requires Zn, which comprises a protein comprising the amino acid sequence of SEQ ID NO: 1, 2, 26, 28, 30, 32, 34, 36, 38, 40, or 42 as an active ingredient.


SEQUENCE LISTING (43 sequences)

SEQ ID NO: 1 - protein, 742 aa, Danio rerio
MMTFLCTRSGRRASGVECRIAAERAYFRVRGLPVANMIGWWPRLCPVMSLALLWACSVGAGSDCKSVAIETDSRIAEQTQQRHLQALFDKYGQNGSISLEGLFNLLKGVGLDRIRKVMVHHPGNAHNHTHTHDHTHTHVDKLTAHTHPVTTKKGDMDHSVEKSDPVPKAQPDPASGKKSQSDAHHNLYMKMNQESTTALTTPSYVTRSRRTNRSADYDFTQDHASFSPSQPNVTHSNHTHHDEDTPTHQHDDHDEHEHARASLGCQNASTILQTHGMRKEASLSVKDFSFLCPALLMQIDSKSCIVHEDEDEHSDHSHHHKHHHHHHDHQHLQHPHNHTNGRGQRNTPVYIAWLGGFLSITLISLLALVGVVLIPLMNRVCFNFLLSFLVALAVGTLSGDALLHLIPHSQGHHHHGHSEEHAEEEDSLRPVWTGLTALSGVYIMFLIEHFLTLGKMYKDKNQKVQKRVDLTTEVLESEKLPSLEENDVKIEAAETNGGRALAEEEEVMLGAELYNDIDCENKCHSHFHDTVGQSDEQHHHHHDYHHILHHHHSQNHHPHTHTHRHTHSYSQQHFEQAGVATLAWMVIMGDGLHNFSDGLAIGAAFTEGLSSGLSTSVAVFCHELPHELGDFAVLLKAGMSVRQAMLYNLLSALMGYLGMIIGILIGHYAENVATWIFALTAGLFMYVALVDMVPEMLHNDASEAGFSHYGFFLLQNAGILLGFGIMLIIAVFEDRIQLDLGY

SEQ ID NO: 2 - protein, 749 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 3 - DNA, 2229 bp, Danio rerio
atgatgacgtttctttgcacacggtctggtcgccgtgctagtggtgtggagtgcagaatcgccgctgaacgcgcttactttcgagtgcgtggactcccggttgccaatatgattggctggtggccacgcctctgcccagtgatgtcactggcactgctgtgggcgtgttcagtgggggcgggttcagactgcaaatctgtggccattgagactgacagccgcatagcagaacaaacacagcagcgtcacctacaggctctgttcgacaagtatggccagaacggcagcatctccctagaaggcctcttcaacctacttaaaggggtcgggcttgaccgcatccggaaagtgatggtgcatcatcctggaaatgcccataatcacacacacacgcatgatcacacacacactcatgtggacaaactcacggcgcacacacatccggtcaccaccaagaagggagacatggatcacagcgtggagaagagtgaccctgtcccaaaagcacagccagatcctgcctctgggaagaaaagccagtcagatgcgcatcacaacctgtacatgaagatgaaccaggaatccaccacagctttgactacgccgtcatatgttaccagatcacggcggaccaatcgcagcgccgattatgattttacacaggaccacgcctcctttagccccagtcagcccaatgtgacacactcaaaccacacccatcatgatgaggacacgcccacacaccagcatgatgaccatgatgagcacgaacatgcccgtgctagtttagggtgtcaaaatgcctccacgatcctgcagacgcatggcatgagaaaggaagcaagtctctcagttaaggacttcagtttcctctgccctgctcttctcatgcagattgattccaagtcttgcatcgtgcatgaagacgaggacgagcattcagatcattcccatcatcacaaacaccaccaccatcatcatgatcaccaacacctgcagcatccacataaccacaccaatggaagaggccagaggaacactccagtctacatcgcatggcttggagggtttctctccatcactctgatcagtttgctggcgttggttggtgtggttttgatcccactcatgaacagagtttgcttcaacttcctgctgagcttcctggtggcccttgcagtgggcactctgagcggagacgctctcctccacctcataccacattctcagggtcatcaccatcacggccactctgaagagcacgctgaagaggaggactcccttcgccctgtgtggaccggactcacagctctaagtggagtttacatcatgttcctcatcgaacacttcctgacccttggcaaaatgtacaaagacaaaaaccagaaggtgcagaagagggttgatctcaccacagaagttttggagtctgagaaactgccatcattagaagaaaatgatgtcaaaattgaagctgctgaaacgaatggtgggcgtgcactggcagaggaggaggaggtgatgttgggggcggagctctacaacgacatagactgcgagaacaaatgccactcccacttccatgacacggtcggccaatcggatgagcagcatcatcatcatcacgactaccaccacatactgcatcatcaccactcccagaaccaccacccgcacacacacacgcacagacacacacactcctactcgcagcagcactttgagcaggctggtgtggccacactcgcctggatggtcatcatgggagacggactgcacaacttcagtgatggacttgccataggggcggctttcacagaaggtttgtccagtggtcttagtacctcagtcgctgtgttctgccatgagcttcctcatgaactcggtgattttgccgtcctactgaaagccggtatgtcagttcgacaggccatgctgtataatctgctgtcagcactgatgggatatctgggcatgatcatcgggattctcatcggacattatgctgaaaatgttgccacatggatctttgctctcacagctgggttattcatgtacgtcgcgctcgtggacatggtacctgagatgctgcacaatgacgcgagcgaagcaggtttcagtcactacggcttcttcctcctgcagaacgctgggatactcctaggcttcggcatcatgcttatcattgctgtctttgaggacaggatccaactggacttaggttactga

SEQ ID NO: 4 - DNA, 2744 bp, Homo sapiens
ctcgtgccgaattcggcacgagaccgcgtgttcgcgcctggtagagatttctcgaagacaccagtgggcccgtgtggaaccaaacctgcgcgcgtggccgggccgtgggacaacgaggccgcggagacgaaggcgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactttggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctagttaaggtttaaatgctagagtagcttaaaaagttgtcatagtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtattaccagtttattatgtaaacaagagatttggcatgacatgttctgtatgtttcagggaaaaatgtctttaatgctttttcaagaactaacacagttattcctatactggattttaggtctctgaagaactgctggtg

SEQ ID NO: 5 - DNA, 25 bp, Artificial
  antisense sequence
cggaaacagcgcgagtgtcttttgt

SEQ ID NO: 6 - DNA, 25 bp, Artificial
  antisense sequence
accgtgtgcaaagaaacgtcatcat

SEQ ID NO: 7 - DNA, 25 bp, Artificial
  antisense sequence
cgcaaaaagcgagagtgtattatgt

SEQ ID NO: 8 - DNA, 25 bp, Artificial
  antisense sequence
acggtctgcaaacaaacgtgatgat

SEQ ID NO: 9 - DNA, 25 bp, Artificial
  antisense sequence
ctcaaggtttcagataaatcgtcct

SEQ ID NO: 10 - DNA, 25 bp, Artificial
  antisense sequence
gccatgttgaccccttaatgtgtcg

SEQ ID NO: 11 - DNA, 25 bp, Artificial
  antisense sequence
ctctaggattcagataaaacgtgct

SEQ ID NO: 12 - DNA, 25 bp, Artificial
  antisense sequence
gccttgtagaccccttaaagtgacg

SEQ ID NO: 13 - DNA, 25 bp, Artificial
  antisense sequence
gtccactccagttactttcagggat

SEQ ID NO: 14 - DNA, 23 bp, Artificial
  antisense sequence
catgctgaactctgaagttgatc

SEQ ID NO: 15 - protein, 58 aa, Danio rerio
AWMVIMGDGLHNFSDGLAIGAAFTEGLSSGLSTSVAVFCHELPHELGDFAVLLKAGMS

SEQ ID NO: 16 - protein, 58 aa, Homo sapiens
AWMVIMGDGLHNFSDGLAIGAAFTEGLSSGLSTSVAVFCHELPHELGDFAVLLKAGMT

SEQ ID NO: 17 - protein, 58 aa, Homo sapiens
AWMVIMGDGIHNFSDGLAIGAAFSAGLTGGISTSIAVFCHELPHELGDFAVLLKAGMT

SEQ ID NO: 18 - protein, 58 aa, Homo sapiens
TWMVLLGDGLHNLTDGLAIGAAFSDGFSAGLSTTLAVFCHELPHELGDFAMLLQSGLS

SEQ ID NO: 19 - protein, 58 aa, Homo sapiens
PYMITLGDAVHNFADGLAVGAAFASSWKTGLATSLAVFCHELPHELGDFAALLHAGLS

SEQ ID NO: 20 - protein, 58 aa, Homo sapiens
AIMILVGDSLHNFADGLAIGAAFSSSSESGVTTTIAILCHEIPHEMGDFAVLLSSGLS

SEQ ID NO: 21 - protein, 58 aa, Homo sapiens
AWMITLCDALHNFIDGLAIGASCTLSLLQGLSTSIAILCEEFPHELGDFVILLNAGMS

SEQ ID NO: 22 - protein, 58 aa, Homo sapiens
AWMITLSDGLHNFIDGLAIGASFTVSVFQGISTSVAILCEEFPHELGDFVILLNAGMS

SEQ ID NO: 23 - protein, 58 aa, Homo sapiens
GYLNLLANTIDNFTHGLAVAASFLVSKKIGLLTTMAILLHEIPHEVGDFAILVQSGCS

SEQ ID NO: 24 - protein, 58 aa, Homo sapiens
GYLNLAADLAHNFTDGLAIGASFRGGRGLGILTTMTVLLHEVPHEVGDFAILLRAGFD

SEQ ID NO: 25 - DNA, 2110 bp, Homo sapiens
  CDS (56)..(1465)
gcgggactgccacgtccaagcaaaccgggaaaggagaggatcccggagccgcgtgatggccagaggcctgggggccccccactgggtggccgtgggactgctgacctgggcgaccttggggcttctggtggctggactcgggggtcatgacgacctgcacgacgatctgcaagaggacttccatggccacagccacaggcactcacatgaagatttccaccatggccacagccatgcccatggccatggccacactcacgagagcatctggcatggacatacccacgatcacgaccatggacattcacatgaggatttacaccatggccatagccatggctactcccatgagagcctctaccacagaggacatggacatgaccatgagcatagccatggaggctatggggagtctggggctccaggcatcaagcaggacctggatgctgtcactctctgggcttatgcactgggggccacagtgctgatctcagcagctccattttttgtcctcttccttatccccgtggagtcgaactctccccggcatcgctctctacttcagatcttgctcagttttgcttccggtgggctcctgggagatgctttcctgcacctcattcctcatgctcttgaacctcattctcaccacactctggagcaacccggacatggacactcccacagtggccagggccccattctgtctgtgggactgtgggttctcagtggaattgttgcctttcttgtcgtggagaaatttgtgagacatgtgaaaggaggacatggtcacagtcatggacatggacacgctcacagtcatacacgtggaagtcatggacatggaagacaagagcgttctaccaaggagaagcagagctcagaggaagaagaaaaggaaacaagaggggttcagaagaggcgaggagggagcacagtacccaaagatgggccagtgagacctcagaacgctgaagaagaaaaaagaggcttagacctgcgtgtgtcggggtacctgaatctggctgctgacttggcacacaacttcactgatggtctggccattggggcttcctttcgagggggccggggactagggatcctgaccacaatgactgtcctgctacatgaagtgccccacgaggtcggagactttgccatcttggtccagtctggctgcagcaaaaagcaggcgatgcgtctgcaactactgacagcagtaggggcactggcaggcacagcctgtgcccttctcactgaaggaggagcagtgggcagtgaaattgcaggtggtgcaggtcctggctgggtcctgccatttactgcaggtggctttatctacgtagcaacagtgtctgtgttgcccgagctgctgagggaggcatcaccattgcaatcacttctggaggtgctggggctgctggggggagttatcatgatggtgctgattgcccaccttgagtgaggggtggataaactacccctgccccaaacctctacccctaactccaggtcaggggtgcgtagaggttgggggccctggccagggacatctgccaaaggaaggaactgtagcctgggagaatggttactttggcattagggccttcaagggctggcagtcttacagaggctggagcggtgagaatgagaggccagagggaccatagtgttgggcactgtctgaccatgttgcatttggaaggctaaatggggccatgaagaaggctggaagggacagggggtgatggcagcctacctggtgtcccctaccccacctgttctcggagaaccaagttgctacacaggaagttctccaaggtccagtttcctttctcccaccagttggtggaggcttcagggaagaccagagtcctggacagagagggtaacaggaggagtcggggataaacatcaaacatcaatcgtgtgtcctgatttgggagtgattggggggatggggtgggagagggttagttggtattctcatggcctgattttttttgtttctattccttttatatcactgtgtttgaatcgagggggaggggtggtaaccggaaataaagacctccgatcttccgccccaaaaaaaaaaaaaaaaaaaaaa

SEQ ID NO: 26 - protein, 469 aa, Homo sapiens
MARGLGAPHWVAVGLLTWATLGLLVAGLGGHDDLHDDLQEDFHGHSHRHSHEDFHHGHSHAHGHGHTHESIWHGHTHDHDHGHSHEDLHHGHSHGYSHESLYHRGHGHDHEHSHGGYGESGAPGIKQDLDAVTLWAYALGATVLISAAPFFVLFLIPVESNSPRHRSLLQILLSFASGGLLGDAFLHLIPHALEPHSHHTLEQPGHGHSHSGQGPILSVGLWVLSGIVAFLVVEKFVRHVKGGHGHSHGHGHAHSHTRGSHGHGRQERSTKEKQSSEEEEKETRGVQKRRGGSTVPKDGPVRPQNAEEEKRGLDLRVSGYLNLAADLAHNFTDGLAIGASFRGGRGLGILTTMTVLLHEVPHEVGDFAILVQSGCSKKQAMRLQLLTAVGALAGTACALLTEGGAVGSEIAGGAGPGWVLPFTAGGFIYVATVSVLPELLREASPLQSLLEVLGLLGGVIMMVLIAHLE

SEQ ID NO: 27 - DNA, 5231 bp, Homo sapiens
  CDS (79)..(2574)
cacgatttggtgcagccggggtttggtaccgagcggagaggagatgcacacggcactcgagtgtgaggaaaaatagaaatgaaggtacatatgcacacaaaattttgcctcatttgtttgctgacatttatttttcatcattgcaaccattgccatgaagaacatgaccatggccctgaagcgcttcacagacagcatcgtggaatgacagaattggagccaagcaaattttcaaagcaagctgctgaaaatgaaaaaaaatactatattgaaaaactttttgagcgttatggtgaaaatggaagattatccttttttggtttggagaaacttttaacaaacttgggccttggagagagaaaagtagttgagattaatcatgaggatcttggccacgatcatgtttctcatttagatattttggcagttcaagagggaaagcattttcactcacataaccaccagcattcccataatcatttaaattcagaaaatcaaactgtgaccagtgtatccacaaaaagaaaccataaatgtgatccagagaaagagacagttgaagtgtctgtaaaatctgatgataaacatatgcatgaccataatcaccgcctacgtcatcaccatcgtttgcatcatcatcttgatcataacaacactcaccattttcataatgattccattactcccagtgagcgtggggagcctagcaatgaaccttcaacagagaccaataaaacccaggaacaatctgatgttaaactaccgaaaggaaagaggaagaaaaaagggaggaaaagtaatgaaaattctgaggttattacaccaggttttccccctaaccatgatcagggtgaacagtatgagcataatcgggtccacaaacctgatcgtgtacataacccaggtcattctcatgtacatcttccagaacgtaatggtcatgatcctggtcgtggacaccaagatcttgatcctgataatgaaggtgaacttcgacatactagaaagagagaagcaccacatgttaaaaataatgcaataatttctttgagaaaagatctaaatgaagatgaccatcatcatgaatgtttgaacgtcactcagttattaaaatactatggtcatggtgccaactctcccatctcaactgatttatttacatacctttgccctgcattgttatatcaaatcgacagcagactttgtattgagcattttgacaaacttttagttgaagatataaataaggataaaaacctggttcctgaagatgaggcaaatataggggcatcagcctggatttgtggtatcatttctatcactgtcattagcctgctttccttgctaggcgtgatcttggttcctatcattaaccaaggatgcttcaaattccttcttacattccttgttgcattagctgtaggaacaatgagtggagacgcccttcttcatctactgccccattctcagggtggacatgatcacagtcaccaacatgcacatgggcatggacattctcatggacatgaatctaacaagtttttggaagaatatgatgctgtattgaaaggacttgttgctctaggaggcatttacttgctatttatcattgaacactgcattagaatgtttaagcactacaaacaacaaagaggaaaacagaaatggtttatgaaacagaacacagaagaatcaactattggaagaaagctttcagatcacaagttaaacaatacaccagattctgactggcttcaactcaagcctcttgccggaactgatgactcggttgtttctgaagatcgacttaatgaaactgaactgacagatttagaaggccaacaagaatcccctcctaaaaattacctttgtatagaagaggagaaaatcatagaccattctcacagtgatggattacataccattcatgagcatgatctccatgctgctgcacataaccaccacggcgagaacaaaactgtgctgaggaagcataatcaccagtggcaccacaagcattctcatcattcccatggcccctgtcattctggatccgatctgaaagaaacaggaatagctaatatagcctggatggtgatcatgggggatggcatccacaacttcagtgatgggctcgcaattggtgcagctttcagtgctggattgacaggaggaatcagtacttctatagccgtcttctgtcatgaactgccacatgaattaggagattttgcagttcttcttaaagcaggcatgactgtaaagcaagcaattgtatacaacctcctctctgccatgatggcttacataggcatgctcataggcacagctgttggtcagtatgccaataacatcacactttggatctttgcagtcactgcaggcatgttcctctatgtagccttggtggatatgcttccagaaatgttgcatggtgatggtgacaatgaagaacatggcttttgtcctgtggggcaattcatccttcagaatttaggattgctctttggatttgccattatgctggtgattgccctctatgaagataaaattgtgtttgacatccagttttgacctttcccagtaatcactgttgattacgagaatgttaccatgcagctttgcatctgttccttgtactgtatgcacattgctcaaaggaaagtcagtggcttgcactacttacaagtttcatagatttgagcctaaccacaagaggctggtgcttagtactgttttccctgcacgtaggggtcttttaaaaatataaagcttgtgataaagagaggagaatatgggactccatgaaccagtgttgatatgtttgattaagacttttcacaaaataatcatataaaacactagtctctttattagtagaaacttctgtggctatgcagaaatagagatcgaaccaaaaaaaatcatttaaactttaaaaatattttaaatggactttggggagacattttttgtgtgttttaagaatgaattgtagtgctctttaattcagctacatatattcatgtggtgatagggatcaacttgacacaactttgaaactgcataaagtagacataggaactagaggaaagctcaggctgcattagagtatgaatttagcattgggaaaagcccttattcttgaatctagagttactatttttgtatatatttgcatagtgtttaaacctgcagcctaaactactgaaatttgtgattgtatgtttgtgtgagcttcagtttaatgaaagattcataatggttctttgtattattataatacttggtgttggggtgttctttctgttttgttttttactttaattttgttttgatttttttttttttttttttggcgggggtaggtgagggtttggagcatgtggtctttttaaaaaattgtaaccctctagaaaatatcaaagaaatgaaccagacgtggtttaaatagttgattttcctattttaacagtaccaactagttaattgggaaatgtaagttctgaatgttcacattgctttaccagtttggcactggaaccaagagcacatgtcgtggctggctacaaggttgtaaagcagaaaatcgaagtttaccatgtctgtaatgtgtacatgaagtgtcaatttagaacagttactaggataaactccattattgccatggctgtcatggtacccaagtgacttggaagatgcatttaaattactcagctgaaatcacttgatcatcttgtgccaagatatgctgttggtgcctgatagggattagtcttttaggtgccctgttctcctaccataattgtgaatgatttgtgagaagtgcaagccatgtttatcctgaatttttacttaataatttgtattactagtcatatgcatgtagctttctgtttacatcctatgccacatggtcttcatttatgccaggtaaactgtatttgaactatgtgcagctagctttgttttaatctgcttggcaaccagtgtagctgctgtaacaatctatcttattgttcaaatatataagagccaaactcttttccattccatctaaaatgttttcatttagtactcttctttcctcctactctatgaacttcaaaacaaaaacaaaactttgagagcagcacatgcatccaggtatttatagattattgccagtgtcttttctgtatgctataagcaagggagcttaggtgttatttctttaatttatgcttgaatctgaaaaattatttctgacttactccatggcctccttataataagtagaagttttatatataattaattttcagcattgggcactgaattaggacagtcctcatctcattgcttggcccttcaagcaacctagctaaaaggtgctgatattttatttagtactgccaacttcaagtgatttagatatctatctatctagatttctgaaccaagatatatttatagttcacttttgggtttttatacccacggtaggattctgcattccagcattaaatctgcttcattttagaacctttataaaagcaatagctggaatatactcccagttttaaaataaatgcctgattgatttaaagcaagtaggttatgctgaagtatataaagaagttttatattctctcaaaaatggtattatctttctttatttgctagattcttacaaatcttttaagagggctgtaacagttgctgctagtattagggttccacatcattctaatgtatagtttcaagtcttaatagacaatctgaattccactacatttcttttggctccaacattccttttagcttgaccagtctaatttaaaatgtgtttgttggaggtcattaacgttacttgtacaatgctgtcactgtgtgacatccatatgaattttggtatatatcaatcaatcaatcaatcacattgcattcaatcaatcagctgtgattgattgattatgcttagaaatactatagtaactagatgcagtgtgaattttttccattaacaaacaaacaaacaagtcagtggcttaaatgtgattatggtcctgcaaggtgattcttgctaaaatatctaaacttttgttttgttttaactgaatcattttttaacttaaaaagctggaaaatatcaaatgctgtttttttttttcattgtcaacagtggtgtgtcattttatgtatgttcctaatgcttatggaactcctccaaaataaagttactcaaagag

SEQ ID NO: 28 - protein, 831 aa, Homo sapiens
MKVHMHTKFCLICLLTFIFHHCNHCHEEHDHGPEALHRQHRGMTELEPSKFSKQAAENEKKYYIEKLFERYGENGRLSFFGLEKLLTNLGLGERKVVEINHEDLGHDHVSHLDILAVQEGKHFHSHNHQHSHNHLNSENQTVTSVSTKRNHKCDPEKETVEVSVKSDDKHMHDHNHRLRHHHRLHHHLDHNNTHHFHNDSITPSERGEPSNEPSTETNKTQEQSDVKLPKGKRKKKGRKSNENSEVITPGFPPNHDQGEQYEHNRVHKPDRVHNPGHSHVHLPERNGHDPGRGHQDLDPDNEGELRHTRKREAPHVKNNAIISLRKDLNEDDHHHECLNVTQLLKYYGHGANSPISTDLFTYLCPALLYQIDSRLCIEHFDKLLVEDINKDKNLVPEDEANIGASAWICGIISITVISLLSLLGVILVPIINQGCFKFLLTFLVALAVGTMSGDALLHLLPHSQGGHDHSHQHAHGHGHSHGHESNKFLEEYDAVLKGLVALGGIYLLFIIEHCIRMFKHYKQQRGKQKWFMKQNTEESTIGRKLSDHKLNNTPDSDWLQLKPLAGTDDSVVSEDRLNETELTDLEGQQESPPKNYLCIEEEKIIDHSHSDGLHTIHEHDLHAAAHNHHGENKTVLRKHNHQWHHKHSHHSHGPCHSGSDLKETGIANIAWMVIMGDGIHNFSDGLAIGAAFSAGLTGGISTSIAVFCHELPHELGDFAVLLKAGMTVKQAIVYNLLSAMMAYIGMLIGTAVGQYANNITLWIFAVTAGMFLYVALVDMLPEMLHGDGDNEEHGFCPVGQFILQNLGLLFGFAIMLVIALYEDKIVFDIQF

SEQ ID NO: 29 - DNA, 2744 bp, Homo sapiens; identical to SEQ ID NO: 4
  CDS (138)..(2387)

SEQ ID NO: 30 - protein, 749 aa, Homo sapiens; identical to SEQ ID NO: 2

SEQ ID NO: 31 - DNA, 4627 bp, Homo sapiens
  CDS (147)..(1625)
ccccggacgcagtgaggggggtcggcgcgcgtgtctacgcggacgcaccggctaagctgcttctgccgccgccggccgcctgggaccttgcggtgaggctgcgcggggccgaggccgcctccgagcgccaggtttattcagtcaccatgaagctgctgctgctgcacccggccttccagagctgcctcctgctgaccctgcttggcttatggagaaccacccctgaggctcacgcttcatccctgggtgcaccagctatcagcgctgcctccttcctgcaggatctaatacatcggtatggcgagggtgacagccccactctgcagcagctgaaggccctactcaaccacctggatgtgggagtgggccggggtaatgtcacccagcacgtgcaaggacacaggaacctctccacgtgctttagttctggagacctcttcactgcccacaatttcagcgagcagtcgcggattgggagcagcgagctccaggagttctgccccaccatcctccagcagctggattcccgggcctgcacctcggagaaccaggaaaacgaggagaatgagcagacggaggaggggcggccaagcgctgttgaagtgtggggctttggttttctcagtgtctcactgattaacctggcctctctcctgggagtcctcgtcctgccctgcacagagaaagcgtttttcagccgtgtgctcacttacttcatcgccctgtccattggaacgctgctgtctaacgcgctattccagctcatcccagaggcatttggtttcaaccctctggaagattattatgtctccaagtctgcagtggtgtttgggggcttttatcttttctttttcacagagaagatcttgaagattcttcttaagcagaaaaatgagcatcatcatggacacagccattatgcctctgagtcgcttccctccaagaaggaccaggaggagggggtgatggagaagctgcagaacggggacctggaccacatgattcctcagcactgcagcagtgagctggacggcaaggcgcccatggtggacgagaaggtcattgtgggctcgctctctgtgcaggacctgcaggcttcccagagtgcttgctactggctgaaaggtgtccgctactctgatatcggcactctggcctggatgatcactctgagcgacggcctccataatttcatcgatggcctggccatcggtgcttccttcactgtgtcagttttccaaggcatcagcacctcggtggccatcctctgtgaggagttcccacgtgagctaggagactttgtcatcctgctcaacgctgggatgagcatccaacaagctctcttcttcaacttcctttctgcctgctgctgctacctgggtctggcctttggcatcctggccggcagccacttctctgccaactggatttttgcgctagctggaggaatgttcttgtatatttctctggctgatatgttccctgagatgaatgaggtctgtcaagaggatgaaaggaagggcagcatcttgattccatttatcatccagaacctgggcctcctgactggattcaccatcatggtggtcctcaccatgtattcaggacagatccagattgggtagggctctgccaagagcctgtgggactggaagtcgggccctgggctgcccgatcgccagcccgaggacttaccatccacaatgcaccacggaagaggccgttctatgaaaaactgacacagactgtattcctgcattcaaatgtcagccgtttgtaaaatgctgtatcctaggaataagctgccctggtaaccagtctctagctagtgcctcttgccctctcctcacctccttttctctcagtgactctggaacctgaatgcagcttacaagacaagcctgacttttttctctgattaccttggcctcctcttggaaccagtgctgaaaggttttgaatcctttacccaacaatgcaaaaatagagccaatggttataacttggctagaaatatcaagagttgaatccatagtgtggggcccatgactctagctgggcaccttggacctccagctggccaatagaagagacaggagacaggaagccttcccattttttcaaagtctgtttaattgcctattacttctctcaaagagaacctgaagtcagaacacatgagcagggtgagaggtgaggcaaggttcatcctgaatgggagaggaagtcgaaccactgctgtgtgtcttgtcaggatgctcacttgttcctactgagatgctggatattgattttgtaacagcacctggtgtttcacggctgtccgagtgagctaacgtggcggtgtggctgcctggacctcctctttcaggttaacgctgacagaatggaggctcaggctgtctgcaagaaaacagttggtttggctgtgattttgacctcctcttccccactgccatcttctaagagactttgtagctgcctcctagaagcacattctgagcacatttgagacctctgtgttagaggggagactgcacaaactatcctcccccaggttgagacgtctgcagagtggcaagctgacttgtagaaatggggtgccatttatgctctacttagacaagggtaatcagaaatggaatcagtgcaggcaaaatttaggatttgccgcttccataaatcaaagcatgactaatagggggtctctgaaatgtaagggcacaaacttcacttagggcatcgcagatgtttgcagaatggttggcctaatgattatgctacagatgggttttaaatgacccgtctaggttactgcttccttgcaaaaaagtcgaatcctgcattgaattgaatatgaatttctctaactctctccagaaaatggatggagataacttgtctttaaaactgtaggccagccttggccactgtggagcccttgcctccgagctctggcttcaaggggagctcttctccaggttcactaggtgaattgatttattattatcatattgataatgtgagattctttagccactttggggagcctgtccctccagaagcctttcttagtggtgcccacagttggagcccaggggccatgtttgcaaactgattcatgtgcatggctgacaggagtactggttcactaccaatgcctgagcttttctcttacatagaaaaactgtccgctctcagtaatcacaagcagcatccgttttgttttctcttcttgggagacatctgtcaaaccaggaatattcttgaaaagaacgtgagcaggaaaaactgctggtgatactttttttaagttttgtttttatcttgcctgttggcttcaatacatttgagaatacgctgaagagggaaaatttcagtgatggagattctagattaaatatcaggactgatttcctggtgggattatggtccagttttaccaaagaaccaattccttgaatgttggaatctaactttttatattgtcattattattgttgtttttaaacggttctttgtcttttctgttttatttttctcaagctgctttcaggagctagcagaaaataactcaaagttgaagactctggaagattttgctttaacctaactcgcattgatgtattaaatttataattttagcattcccaatagatcctatcattccttaaacataataccctttgtcttggagtagaatactaagttagagttagtggatttctagtttaggagaggagctcaaaactataatctttaacaaattgaaaaatgaaatagggtgttttccctttttgtgcacacctatattaccttaagaaatttccttccatagacagctgcctcaaagggaaatcctctttaaaccgtagttggcgcagaggtcagtcctagtcggagcttaggaggggcggagacgctcacatcgtctgacttgagtcgccactgattgtggcaacagctttgcctcatgagtcaaaaattggcaatttcttttgatttttagttgttgaatttgctgtttcaagcatttgtacatattagaagtctaaggagtagcaagtcagtgggaggactttttcacccctggcattagcagcttcgacctcattttccagatgcaccagctcctattaataagttagcaaggaaagtgtatgtcacgtgcaggaacagtgaggcagggacaggggttctgctccttctcacttcaccaccggcacacagcttgcccctgtctttgcccccaaaggtattttgtgtctagtgtcaaattggagctattcttcactggtccttaaccttgggttttaaaaagaaggcttctctgtttgggtagcgtaagagctgagtatagtaagtcctcttccaaagagatggcaatatgctgggcatctactttaaaacaaagttgtctgatttttgcaagagaggttaggattttattgttcttatttccctttacagttctgcagttccatcacagtatttttttaaataactcaggtgtatgagaagaaattagaaaagaaaattaacttatgtggactgtaaatgttttatttgtaagattctataaataaagctatattctgtaattgttgagaaaaaaaaaaaaaaaaaa

SEQ ID NO: 32 - protein, 492 aa, Homo sapiens
MKLLLLHPAFQSCLLLTLLGLWRTTPEAHASSLGAPAISAASFLQDLIHRYGEGDSPTLQQLKALLNHLDVGVGRGNVTQHVQGHRNLSTCFSSGDLFTAHNFSEQSRIGSSELQEFCPTILQQLDSRACTSENQENEENEQTEEGRPSAVEVWGFGFLSVSLINLASLLGVLVLPCTEKAFFSRVLTYFIALSIGTLLSNALFQLIPEAFGFNPLEDYYVSKSAVVFGGFYLFFFTEKILKILLKQKNEHHHGHSHYASESLPSKKDQEEGVMEKLQNGDLDHMIPQHCSSELDGKAPMVDEKVIVGSLSVQDLQASQSACYWLKGVRYSDIGTLAWMITLSDGLHNFIDGLAIGASFTVSVFQGISTSVAILCEEFPRELGDFVILLNAGMSIQQALFFNFLSACCCYLGLAFGILAGSHFSANWIFALAGGMFLYISLADMFPEMNEVCQEDERKGSILIPFIIQNLGLLTGFTIMVVLTMYSGQIQIG

SEQ ID NO: 33 - DNA, 2178 bp, Homo sapiens
  CDS (87)..(2030)
caaccccagcaaagccgccctcagccagcccagaagcactgggccttggccacagcaacacccactgagcacgctgggagctgagtatggcgtccctggtctcgctggagctggggctgcttctggctgtgctggtggtgacggcgacggcgtccccgcctgctggtctgctgagcctgctcacctctggccagggcgctctggatcaagaggctctgggcggcctgttaaatacgctggcggaccgtgtgcactgcaccaacgggccgtgtggaaagtgcctgtctgtggaggacgccctgggcctgggcgagcctgaggggtcagggctgcccccgggcccggtcctggaggccaggtacgtcgcccgcctcagtgccgccgccgtcctgtacctcagcaaccccgagggcacctgtgaggacactcgggctggcctctgggcctctcatgcagaccacctcctggccctgctcgagagccccaaggccctgaccccgggcctgagctggctgctgcagaggatgcaggcccgggctgccggccagacccccaagacggcctgcgtagatatccctcagctgctggaggaggcggtgggggcgggggctccgggcagtgctggcggcgtcctggctgccctgctggaccatgtcaggagcgggtcttgcttccacgccttgccgagccctcagtacttcgtggactttgtgttccagcagcacagcagcgaggtccctatgacgctggccgagctgtcagccttgatgcagcgcctgggggtgggcagggaggcccacagtgaccacagtcatcggcacaggggagccagcagccgggaccctgtgcccctcatcagctccagcaacagctccagtgtgtgggacacggtatgcctgagtgccagggacgtgatggctgcatatggactgtcggaacaggctggggtgaccccggaggcctgggcccaactgagccctgccctgctccaacagcagctgagtggagcctgcacctcccagtccaggccccccgtccaggaccagctcagccagtcagagaggtatctgtacggctccctggccacgctgctcatctgcctctgcgcggtctttggcctcctgctgctgacctgcactggctgcaggggggtcacccactacatcctgcagaccttcctgagcctggcagtgggtgcactcactggggacgctgtcctgcatctgacgcccaaggtgctggggctgcatacacacagcgaagagggcctcagcccacagcccacctggcgcctcctggctatgctggccgggctctacgccttcttcctgtttgagaacctcttcaatctcctgctgcccagggacccggaggacctggaggacgggccctgcggccacagcagccatagccacgggggccacagccacggtgtgtccctgcagctggcacccagcgagctccggcagcccaagcccccccacgagggctcccgcgcagacctggtggcggaggagagcccggagctgctgaaccctgagcccaggagactgagcccagagttgaggctactgccctatatgatcactctgggcgacgccgtgcacaacttcgccgacgggctggccgtgggcgccgccttcgcgtcctcctggaagaccgggctggccacctcgctggccgtgttctgccacgagttgccacacgagctgggggacttcgccgccttgctgcacgcggggctgtccgtgcgccaagcactgctgctgaacctggcctccgcgctcacggccttcgctggtctctacgtggcactcgcggttggagtcagcgaggagagcgaggcctggatcctggcagtggccaccggcctgttcctctacgtagcactctgcgacatgctcccggcgatgttgaaagtacgggacccgcggccctggctcctcttcctgctgcacaacgtgggcctgctgggcggctggaccgtcctgctgctgctgtccctgtacgaggatgacatcaccttctgataccctgccctagtcccccacctttgacttaagatcccacacctcacaaacctacagcccagaaaccagaagcccctatagaggccccagtcccaactccagtaaagacactcttgtccttggaaaaaaaaaaaaaaaaaaaaaaaaa

SEQ ID NO: 34 - protein, 647 aa, Homo sapiens [sequence omitted]

SEQ ID NO: 35 - DNA, 3246 bp, Homo sapiens
  CDS (478)..(1860)
  misc_feature (2530)..(2530): n is a, c, g, or t
agtgtggttttagtttttcctaagaagtggcgtggtttggggctttatatccgggaggagcatatgtacgcaaatcctggggcgtttgcaaacccggatccggggcgtctggccccatgcccggccgggcgtttgagggctactgccacgcagcgtttctggagcctgccggctggtgccctggtggcctttatctctgtccccctttgtcctctttatctcaggctctccaggaggccggggggcccactccgcctatcgctcccctcggctacgctgccactccaatgccccgcaggtcgcgagctgctgttctttcgaaggcgccggagaaccaggggcgtcccgcgccacctctgactcggagcagcgccgagcactgacgctcccgcccttgggcaaggacgccagtgcgcccgcgcgcgtccctctgcgcggcagcccgtcgcgggccctcaaggggaagcccaggccaggatggccccgggtcgcgcggtggccgggctcctgttgctggcggccgccggcctcggaggagtggcggaggggccagggctagccttcagcgaggatgtgctgagcgtgttcggcgcgaatctgagcctgtcggcggcgcagctccagcacttgctggagcagatgggagccgcctcccgcgtgggcgtcccggagcctggccagctgcacttcaaccagtgtttaactgctgaagagatcttttcccttcatggcttttcaaatgctacccaaataaccagctccaaattctctgtcatctgtccagcagtcttacagcaattgaactttcacccatgtgaggatcggcccaagcacaaaacaagaccaagtcattcagaagtttggggatatggattcctgtcagtgacgattattaatctggcatctctcctcggattgattttgactccactgataaagaaatcttatttcccaaagattttgaccttttttgtggggctggctattgggactcttttttcaaatgcaattttccaacttattccagaggcatttggatttgatcccaaagtcgacagttatgttgagaaggcagttgctgtgtttggtggattttacctacttttcttttttgaaagaatgctaaagatgttattaaagacatatggtcagaatggtcatacccactttggaaatgataactttggtcctcaagaaaaaactcatcaacctaaagcattacctgccatcaatggtgtgacatgctatgcaaatcctgctgtcacagaagctaatggacatatccattttgataatgtcagtgtggtatctctacaggatggaaaaaaagagccaagttcatgtacctgtttgaaggggcccaaactgtcagaaatagggacgattgcctggatgataacgctctgcgatgccctccacaatttcatcgatggcctggcgattggggcttcctgcaccttgtctctccttcagggactcagtacttccatagcaatcctatgtgaggagtttccccacgagttaggagactttgtgatcctactcaatgcagggatgagcactcgacaagccttgctattcaacttcctttctgcatgttcctgctatgttgggctagcttttggcattttggtgggcaacaatttcgctccaaatattatatttgcacttgctggaggcatgttcctctatatttctctggcagatatgtttccagagatgaatgatatgctgagagaaaaggtaactggaagaaaaaccgatttcaccttcttcatgattcagaatgctggaatgttaactggattcacagccattctactcattaccttgtatgcaggagaaatcgaattggagtaatagaaaatggaagatggtgttgttaataaaggcatttaatagataaaaacatctccaaaaaggattttgaagctgatcctatttagttaaaaagataattttgctttcaactgtaggtccagaaaactaattattggcatcagtctgtgaaatagtccattatttgttgttaaaaatgcttcaaaaggttttcagtgtcagtctgagatgcctggtatataggagcctttgggaaatacttatttttcagtattccatgcatattagatatcaccatgaagcaagagacatgcattctataatcatgtagacactcagactcaggggaaaatacaagttatatcctgaaagcctttaaaactctatggtaggatcaaagattcaaatggtttcagagaggttttatttcaattaatttgttctagtgctttcaagagcaagtacatcaaaatgtagaaggtaaaatgtatgcaacactaatataaattattccaagtctttaaggagccaaagaaaaaaaagatttctcacagctttttgttctgttttgtatttcaattaggaacttgcagtattattttgaaaaccattctaaaataataggagttaggaaataaataaagttttgctagccctgctaagttcaggcttagaggcttatcgctaagtntaaacttcaccagattccacgaaaagctggatagctttttttctgacttatgttgtggttgcacccctcacaaatggcagaacagtatgtaaagctggtaacacctcggtttcagtgcaccatgtgtttgctttgtgaaggtgaagaatatgttggtttagagaaagaaattggatgtaattttatgcaatttacttttaaagacaaacataactatttagcagagaatattttaataaatgcaaaacaacagctggactgctgtacatcaaggacagattaactggaaaacatatgttccttatgtgtgattgagagccattcagaaaagacttcctttgtgttcagcctatacttttccatatggtataccttgaaaaaaattagcacaccatggttatttttctaccttttataaaagacagagcctgtttactcatttagaagatagagaaaattggtctaaaattgaacatcctagattcacactcccaagtcacttaaggtgatttgatggtgaggaaaatgattgacaaagcccaacaatgatctcaggaattacattttccaacagaccaaaaaatgttttcatgtagcagcaatgcagatttggtgaatatttaatatatattttagtatgtatttcactttatgactgacaattaaaaaatattgtttggccaaatagtaaacacccttttgaaaccatgaaaaaa

SEQ ID NO: 36 - protein, 460 aa, Homo sapiens
MAPGRAVAGLLLLAAAGLGGVAEGPGLAFSEDVLSVFGANLSLSAAQLQHLLEQMGAASRVGVPEPGQLHFNQCLTAEEIFSLHGFSNATQITSSKFSVICPAVLQQLNFHPCEDRPKHKTRPSHSEVWGYGFLSVTIINLASLLGLILTPLIKKSYFPKILTFFVGLAIGTLFSNAIFQLIPEAFGFDPKVDSYVEKAVAVFGGFYLLFFFERMLKMLLKTYGQNGHTHFGNDNFGPQEKTHQPKALPAINGVTCYANPAVTEANGHIHFDNVSVVSLQDGKKEPSSCTCLKGPKLSEIGTIAWMITLCDALHNFIDGLAIGASCTLSLLQGLSTSIAILCEEFPHELGDFVILLNAGMSTRQALLFNFLSACSCYVGLAFGILVGNNFAPNIIFALAGGMFLYISLADMFPEMNDMLREKVTGRKTDFTFFMIQNAGMLTGFTAILLITLYAGEIELE

SEQ ID NO: 37 - DNA, 1895 bp, Homo sapiens
  CDS (106)..(1725)
gtctacagcaaggcctaataggggacctgagggcacagtcctcaggatgtttcggggagaataggagccagaacctgagcccctaagccattcccctcaccaatgatggggtccccagtgagtcatctgctggccggcttctgtgtgtgggtcgtcttgggctgggtagggggctcagtccccaacctgggccctgctgagcaggagcagaaccattacctggcccagctgtttggcctgtacggcgagaatgggacgctgactgcagggggcttggcgcggcttctccacagcctggggctaggccgagttcaggggcttcgcctgggacagcatgggcctctgactggacgggctgcatccccagctgcagacaattccacacacaggccacagaaccctgagctgagtgtggatgtctgggcagggatgcctctgggtccctcagggtggggtgacctggaagagtcaaaggcccctcacctaccccgtgggccagccccctcgggcctggacctccttcacaggcttctgttgctggaccactcattggctgaccacctgaatgaggattgtctgaacggctcccagctgctggtcaattttggcttgagccccgctgctcctctgacccctcgtcagtttgctctgctgtgcccagccctgctttatcagatcgacagccgcgtctgcatcggcgctccggcccctgcacccccaggggatctactatctgccctgcttcagagtgccctggcagtcctgttgctcagcctcccttctcccctatccctgctgctgctgcggctcctgggacctcgtctactacggcccttgctgggcttcctgggggccctggcggtgggcactctttgtggggatgcactgctacatctgctaccgcatgcacaagaagggcggcacgcaggacctggcggactaccagagaaggacctgggcccggggctgtcagtgctcggaggcctcttcctgctctttgtgctggagaacatgctggggcttttgcggcaccgagggctcaggccaagatgctgcaggcgaaaacgaaggaatctcgaaacacgcaacttggatccggagaatggcagtgggatggcccttcagcccctacaggcagctccagagccaggggctcagggccagagggagaagaacagccagcacccaccagctctggcccctcctgggcaccaaggccacagtcatgggcaccagggtggcactgatatcacgtggatggtcctcctgggagatggtctacacaacctcactgatgggctggccataggtgctgccttctctgatggcttctccagcggcctcagtaccaccttagcggtcttctgccatgagctgccccacgaactgggtgactttgccatgctgctccagtcagggctgtcctttcggcggctgctgctgctgagcctcgtgtctggagccctgggattggggggtgcagtcctgggggtggggctcagcctgggccctgtccccctcactccctgggtgtttggggtcactgctggggtcttcctctatgtggcccttgtggacatgctaccagccctgcttcgtcctccggagcccctgcctacgccccatgtgctcctgcaggggctggggctgctgctggggggcggcctcatgcttgccataaccctgctggaggagcggctactgcccgtgaccactgagggctgatggggccagtggaaaggggtcgggttgcccttccttccccccaaccacaggaatggaggcgggacacagggccagtaggagcaataggattttaataaacagaacccatcccaaaaaaaaaaaaaaaaaaaaaaaaaaaaaaaaaaaaaaaaaaaaaaaaaaaaaaaaaa

SEQ ID NO: 38 - protein, 539 aa, Homo sapiens
MGSPVSHLLAGFCVWVVLGWVGGSVPNLGPAEQEQNHYLAQLFGLYGENGTLTAGGLARLLHSLGLGRVQGLRLGQHGPLTGRAASPAADNSTHRPQNPELSVDVWAGMPLGPSGWGDLEESKAPHLPRGPAPSGLDLLHRLLLLDHSLADHLNEDCLNGSQLLVNFGLSPAAPLTPRQFALLCPALLYQIDSRVCIGAPAPAPPGDLLSALLQSALAVLLLSLPSPLSLLLLRLLGPRLLRPLLGFLGALAVGTLCGDALLHLLPHAQEGRHAGPGGLPEKDLGPGLSVLGGLFLLFVLENMLGLLRHRGLRPRCCRRKRRNLETRNLDPENGSGMALQPLQAAPEPGAQGQREKNSQHPPALAPPGHQGHSHGHQGGTDITWMVLLGDGLHNLTDGLAIGAAFSDGFSSGLSTTLAVFCHELPHELGDFAMLLQSGLSFRRLLLLSLVSGALGLGGAVLGVGLSLGPVPLTPWVFGVTAGVFLYVALVDMLPALLRPPEPLPTPHVLLQGLGLLLGGGLMLAITLLEERLLPVTTEG

SEQ ID NO: 39 - DNA, 2636 bp, Homo sapiens
  CDS (222)..(2186)
aaaataatcctctctagctcccagtgcacagtcaaagaattttaaaaacagggaactttgtaactgtgaaatactctccaggatttaaaaggctgtggagctccagataaagaatcgtttatctttcttctgaagaaattcctttggttacaagtttaccccataaacggcaacacactcacctccatccaagacagactcaaggtggaggaagcgtggaaatgtgcttccggacaaagctctcagtatcctgggtgccattgtttcttctactcagccgtgttttttctactgagacagacaaaccctcagcccaggacagcagaagccgtgggagttcaggccaaccggcagacctgctacaggttctctctgctggtgaccacccaccccacaaccactcaagaagcctcatcaaaacattgttggagaaaactgggtgcccacggaggagaaacggaatgcaaggagattgcaatctgtgctttgaaccagatgcactattactaatagctggaggaaattttgaagatcagcttagagaagaagtggtccagagagtttctcttctccttctctattacattattcatcaggaagagatctgttcttcaaagctcaacatgagtaataaagagtataaattttacctacacagcctactgagcctcaggcaggatgaagattcctctttcctttcacagaatgagacagaagatatcttggctttcaccaggcagtactttgacacttctcaaagccagtgtatggaaaccaaaacgctgcagaaaaaatctggaatagtgagcagtgaaggtgctaatgaaagtacgcttcctcagttggcagccatgatcattactttgtccctccagggtgtttgtctgggacaaggaaacttgccttccccagactactttacagaatatattttcagttccttgaatcgtacgaataccctccgcctatcagaactagaccaactcctcaacactctctggaccagaagtacttgtatcaaaaatgagaaaatccatcaatttcaaaggaaacaaaacaacataataacccatgatcagggctattctaatttctcttcatccatggaaaaagagtctgaggatggtccagtttcctgggatcagacctgcttctctgctaggcagctggtggagatatttctacagaagggcctctcactcatttctaaggaggactttaagcaaatgagtccagggatcatccagcagctcctcagctgctcctgccacttacccaaggaccaacaagcaaagctgccacctaccactctggagaaatacggctacagcacggtggctgtcacccttctcacactgggctccatgctggggacagcgctggtccttttccatagctgtgaggagaactacaggcttatcttacagctgtttgtgggcttggccgtcgggacactgtctggggacgctctgctccaccttatccctcaggttcttggtttacataagcaggaagccccagaatttgggcatttccatgaaagcaaaggtcatatttggaaactgatgggattaattggaggcatccatggatttttcttgatagaaaaatgttttattcttcttgtatcaccaaatgacaagaaaagcccagaagattcacaggcagctgaaatgcctataggcagtatgacagcctccaacagaaaatgtaaagccattagcttgttagcaatcatgattctggttggggacagcctgcataattttgcagatggcctagccataggagcagccttctcatcatcatccgagtcaggagtgaccactacgattgctatcttgtgtcatgaaatcccacatgaaatgggagactttgccgtgctcttaagctctggactttctatgaagactgccatcctgatgaattttataagctccctaactgccttcatgggattatacattggcctttccgtgtcagctgatccatgtgttcaagactggatcttcacagtcactgctgggatgttcttatatttatccttggttgaaatgcttcctgaaatgactcatgttcaaacacaacgaccctggatgatgtttctcctgcaaaactttggattgatcctaggttggctttctctcctgctcttggctatatatgagcaaaatattaaaatataagtgaggatcttcaacatctttcaaaaatgcatttatatagtcttactttgtttctttcattgcactctataatgatttttaaattaagaattttttatcttaggcaaagtgtgtctctttcaattcattaacttattaattttataatgcagttttatttttggaaacatataaatatcagactgtccttaattgaaattttgtctttggtttccaacaccatgatgaagctcttgctttttaaaaagtagttagtaaattctgcatgaattttagtaaactttaaaaaatagattttttccctaagaaagaatgtttgtagaatttaaagtggacagatgcctgttggggtaaaatcaactgcaactttttgatgttaatttttttccctgtgcaattataaactataagcaagttaagtgacaagcaaatgtaataaagactagtttt

SEQ ID NO: 40 - protein, 654 aa, Homo sapiens
MCFRTKLSVSWVPLFLLLSRVFSTETDKPSAQDSRSRGSSGQPADLLQVLSAGDHPPHNHSRSLIKTLLEKTGCPRRRNGMQGDCNLCFEPDALLLIAGGNFEDQLREEVVQRVSLLLLYYIIHQEEICSSKLNMSNKEYKFYLHSLLSLRQDEDSSFLSQNETEDILAFTRQYFDTSQSQCMETKTLQKKSGIVSSEGANESTLPQLAAMIITLSLQGVCLGQGNLPSPDYFTEYIFSSLNRTNTLRLSELDQLLNTLWTRSTCIKNEKIHQFQRKQNNIITHDQGYSNFSSSMEKESEDGPVSWDQTCFSARQLVEIFLQKGLSLISKEDFKQMSPGIIQQLLSCSCHLPKDQQAKLPPTTLEKYGYSTVAVTLLTLGSMLGTALVLFHSCEENYRLILQLFVGLAVGTLSGDALLHLIPQVLGLHKQEAPEFGHFHESKGHIWKLMGLIGGIHGFFLIEKCFILLVSPNDKKSPEDSQAAEMPIGSMTASNRKCKAISLLAIMILVGDSLHNFADGLAIGAAFSSSSESGVTTTIAILCHEIPHEMGDFAVLLSSGLSMKTAILMNFISSLTAFMGLYIGLSVSADPCVQDWIFTVTAGMFLYLSLVEMLPEMTHVQTQRPWMMFLLQNFGLILGWLSLLLLAIYEQNIKI

SEQ ID NO: 41 - DNA, 2309 bp, Homo sapiens
  CDS (43)..(1158)
gggccgcccggatcgccgccgccgccgccgcacgtacgtggcatgcctggatgtccctgccctggctgtggcatggcgggcccaaggctcctcttcctcactgcccttgccctggagctcttgggaagggctgggggttcccagccggccctccggagccgggggactgcgacggcctgtcgcctggacaacaaggaaagcgagtcctggggggctctgctgagcggagagcggctggacacctggatctgctccctcctgggttccctcatggtggggctcagtggggtcttcccgttgcttgtcattcccctagagatggggaccatgctgcgctcagaagctggggcctggcgcctgaagcagctgctcagcttcgccctggggggactcttgggcaatgtgtttctgcatctgctgcccgaagcctgggcctacacgtgcagcgccagccctggtggtgaggggcagagcctgcagcagcagcaacagctggggctgtgggtcattgctggcatcctgaccttcctggcgttggagaagatgttcctggacagcaaggaggaggggaccagccaggcccccaacaaagaccccactgctgctgccgccgcactcaatggaggccactgtctggcccagccggctgcagagcccggcctcggtgccgtggtccggagcatcaaagtcagcggctacctcaacctgctggccaacaccatcgataacttcacccacgggctggctgtggctgccagcttccttgtgagcaagaagatcgggctcctgacaaccatggccatcctcctgcatgagatcccccatgaggtgggcgactttgccatcctgctccgggccggctttgaccgatggagcgcagccaagctgcaactctcaacagcgctggggggcctactgggcgctggcttcgccatctgtacccagtcccccaagggagtagttgggtgttctcccgctgcagaggagacggcagcctgggtcctgcccttcacctctggcggctttctctacatcgccttggtgaacgtgctccctgacctcttggaagaagaggacccgtggcgctccctgcagcagctgcttctgctctgtgcgggcatcgtggtaatggtgctgttctcgctcttcgtggattaactttccctgatgccgacgcccctgccccctgcagcaataagatgctcggattcactctgtgaccgcatatgtgagaggcagagagggcgagtggctgcgagagagaatgagcctcccgccagacaggagggaggtgcgtgtggatgtatgtggtgtgcacatgtggccagaggtgtgtgcgcgagaccgacactgtgatccctgtgctgggtccggggcccagtgtagcgcctgtccccagccatgctgtggttacctctccttgccgccctgtcaccttcacctcctggagtaagcagcgaggaagagcagcactggtcccaagcagaggccttgccctgctgggaccccgggagtgagagcagcccaaggatcccagggtgcagggaactccagagctgcccacctcccactgccccctcagcacacacacagtccccaggcggcctaggggccaaggctggggcggctttggtcccttttcctggcccttccttccccacttctaagccaaagaaaggagaggcaggtgctcctgtaccccagccccactcagcactgacagtccccagctcctagtagtgagctgggaggcgcttcctaagaccctttcctcagggctgccctgggagctcattcctggccaacacgccctggcagcaccagcagctcttgccacctccagctgccaaacagcagcctgccgggcagggagcagccccaggccagagaggcctcccggtccagctcagggatgctcctgccagcacaggggccagggactcctggagcaggcacatagtgagcccgggcagccctgcccagctcaggcccctttccttccccattgaggttggggtaggtgggggcggtgagggctccacgttgtcagcgctcaggaatgtgctccggcagagtgctgaagccataatccccaaccatttcccttggctgacgcccaggtactcagctggcccactccacagccaggcctggccctgcccttcaccgtggatgttttcagaagtggccatcgagaggtctggatggttttatagcaactttgctgtgattccgtttgtatctgtaaatatttgttctatagataagatacaaataaatattatccacataaaaaaaaaaaaaaaaaa

SEQ ID NO: 42 - protein, 371 aa, Homo sapiens
MPGCPCPGCGMAGPRLLFLTALALELLGRAGGSQPALRSRGTATACRLDNKESESWGALLSGERLDTWICSLLGSLMVGLSGVFPLLVIPLEMGTMLRSEAGAWRLKQLLSFALGGLLGNVFLHLLPEAWAYTCSASPGGEGQSLQQQQQLGLWVIAGILTFLALEKMFLDSKEEGTSQAPNKDPTAAAAALNGGHCLAQPAAEPGLGAVVRSIKVSGYLNLLANTIDNFTHGLAVAASFLVSKKIGLLTTMAILLHEIPHEVGDFAILLRAGFDRWSAAKLQLSTALGGLLGAGFAICTQSPKGVVGCSPAAEETAAWVLPFTSGGFLYIALVNVLPDLLEEEDPWRSLQQLLLLCAGIVVMVLFSLFVD

SEQ ID NO: 43 - DNA, 19 bp, Artificial
  A target sequence for siRNA
aggagaaagtagatacaga